(19) United States
(12) Patent Application Publication
(10) Pub. No.: US 2013/0345073 A1
(43) Pub. Date: Dec. 26, 2013

(54) USE OF ALPHA-2-MACROGLOBULIN FOR DETERMINING OSTEONECROSIS DISEASE STATUS AND FOR SCREENING THERAPEUTIC AGENTS FOR PREVENTING OR TREATING OSTEONECROSIS

(71) Applicants: Chantal Séguin, Montreal (CA); Edward Harvey, Westmount (CA); Denis Cournoyer, Montreal (CA); Terry Y. Chow, Anjou (CA); Mohammad Amin Kerachian, Candiac (CA)

(72) Inventors: Chantal Séguin, Montreal (CA); Edward Harvey, Westmount (CA); Denis Cournoyer, Montreal (CA); Terry Y. Chow, Anjou (CA); Mohammad Amin Kerachian, Candiac (CA)

(21) Appl. No.: 13/530,342

(22) Filed: Jun. 22, 2012

(51) Int. Cl. C40B 30/04; G01N 33/573; G01N 33/566; C12Q 1/68; G01N 21/64

(52) U.S. Cl. 506/9; 435/6.13; 435/6.12; 435/7.1; 435/7.4

(57) ABSTRACT

The present application presents a method of assessing a predisposition to osteonecrosis in an individual based on the level of expression/activity of alpha-2-macroglubulin, a biomarker whose expression is positively correlated with osteonecrosis. Also provided herein are a diagnostic/prognostic system as well as a software product based on the determination of the level of expression/activity of alpha-2-macroglobulin. Screening methods for the determination of usefulness of an agent in the prevention and/or treatment of osteonecrosis are also provided.

### TECHNOLOGICAL FIELD

This application relates to alpha-2-macroglobulin as a marker for the assessment of predisposition of osteonecrosis as well as for the determination of usefulness of therapeutic agents in the treatment and/or prevention of osteonecrosis

### BACKGROUND

Osteonecrosis (ON) or avascular necrosis of the femoral head (ANFH or AVN) is a disabling and progressive condition in young patients, which leads to femoral head collapse and eventual total hip arthroplasty. Numerous conditions have been implicated in ON. Unfortunately, there is currently no biomarker to evaluate the activity status or the prognosis of the disease. The pathogenesis of “idiopathic” ON is incompletely understood and therefore predictors of disease initiation or progression are lacking. Two major limitations in the past have impeded the delineation of the pathophysiology: a lack of understanding of the interaction between the disease and the coagulation abnormalities and a lack of suitable animal models. Currently, amongst several pathogenic mechanisms, the vascular hypothesis, (or regional endothelial bed dysfunction) in which local microvascular thrombosis leads to a decrease in blood flow in the femoral head, has become more accepted. The fact that ON is sometimes seen in twins and in familial clusters suggests that genetic factors are also involved. New evidence of increased incidence of ON in specific animal models provides further evidence of genetic susceptibility. Although observed systemic thrombophilic and hypofibrinolytic coagulation abnormalities in patients with ON is increased in some studies compared to controls, the vast majority of ON patients do not demonstrate significant differences in the levels of thrombotic and fibrinolytic factors.

The current pathophysiological model of ON postulates a multiple hit theory such that with increasing number of risk factors the chance of ON increases. Amongst the many risk factors, glucocorticosteroids (GCs) play the leading role in non-traumatic cases of ON. Even when GCs are thought to be the cause, a careful history is suggested to identify other risk factors. GCs are the mainstay of therapy in most inflammatory disorders and they are also included in most chemotherapy protocols. Therefore, ON is thus a potential major complication for large patient populations. Investigators have proposed both direct and indirect effects of GCs on cells. Indirect and direct mechanisms remain intimately related and often result in positive feedback loops to potentiate the disease processes. However, the direct effects, in particularly apoptosis, have recently been shown to be increasingly important. Suppression of osteoblast and osteoclast precursor production, increased apoptosis of osteoblasts and osteocytes, prolongation of the lifespan of osteoclasts and apoptosis of endothelial cells (EC) are all direct effects of GC usage.

Since osteonecrosis cannot be easily diagnosed or easily treated when diagnosed in the late stages of the disease, it would be highly desirable to be provided with a biomarker that is associated with the predictability of onset of osteonecrosis.

### SUMMARY

The present application provides a method of determining the risk of developing or the presence of osteonecrosis in an individual, based on alpha-2-macroglobulin (A2M) levels, which are shown herein to be increased with the disease predisposition and onset. The present application also provides a screening method for agents for the prevention or treatment of osteonecrosis based on the ability of the agent to modulate the level of A2M levels.

According to a first aspect, the present invention provides a method of assessing a predisposition to or an affliction by osteonecrosis in an individual. Broadly, the method comprises obtaining, from a biological sample of the individual, a level of a parameter of at least one biomarker whose expression is positively correlated with osteonecrosis, wherein said at least one biomarker comprises alpha-2-macroglobulin (A2M); comparing the level of the parameter of the at least one biomarker to a control level of the parameter of the at least one biomarker, wherein the control level is associated with a lack of osteonecrosis; and characterizing the individual. The individual is characterized as being susceptible to or afflicted by osteonecrosis when the level of the parameter of the at least one biomarker is higher than the control level of the parameter of the at least one biomarker; and lacking the susceptibility or the affliction to osteonecrosis when the level of the parameter of the at least one biomarker is equal to or lower than the control level of the parameter of the at least one biomarker. In an embodiment, the at least one biomarker further comprises collagen type II alpha-1 (col2A1). In another embodiment, the at least one biomarker further comprises melanoma inhibitory activity 1 (MIA1). In still another embodiment, the at least one biomarker further comprises a steroid stimulus response gene selected from the group consisting of alkaline phosphatase, tissue-nonspecific, transforming growth factor beta 2 and potassium large conductance calcium-activated channel, subfamily m, alpha member 1. In still another embodiment, the at least biomarker further comprises an apoptosis pathway response gene selected from the group consisting of S100 protein-beta polypeptide, transforming growth factor-beta 2, vitamin D receptor, unc-5 homolog c and growth hormone receptor. In yet a further embodiment, the at least one biomarker further comprises scrapie responsive gene 1, growth hormone receptor, SH2B adaptor protein 2, fibromodulin, matrix metallopeptidase 3, Proprotein convertase subtilisin/kexin type 6, cadherin 13, calpain 6, murinoglobulin 2, solute carrier family 38, member 3, fibroblast growth factor 1, vitamin D receptor, carbonic anhydrase 8, WNT1 inducible signaling pathway protein 2, integrin binding sialoprotein, calcitonin receptor, S100 protein, beta polypeptide, neural, potassium large conductance calcium-activated channel, subfamily M, alpha member 1, angiopoietin-like 2, pannexin 3, sphingomyelin phosphodiesterase 3, neutral solute carrier family 13 (sodium-dependent citrate transporter) member 5, cadherin 17, unc-5 homolog C, plasminogen activator inhibitor-1, solute carrier organic anion transporter family, member 2a1, melanoma cell adhesion molecule, orosomucoid 1, transforming growth factor beta 2, alkaline phosphatase liver/bone/kidney, basic helix-loop-helix domain containing class B3, immunoglobulin superfamily member 10, transmembrane protein 100. parathyroid hormone receptor 1, a disintegrin-like and metallopeptidase (reprolysin type) with thrombospondin type 1 motif 1, sphingomyelin synthase 2, transient receptor potential cation channel, subfamily V member 4, parvin alpha, regulator of calcineurin 2, latexin, receptor accessory protein 6 and/or cAMP responsive element binding protein 3-like 1. Optionally, the method can further comprise obtaining a level of a parameter of at least one biomarker whose expression is negatively correlated with osteonecrosis in the biological sample from the individual, wherein said at least one biomarker is chemokine (C-X-C motif) ligand 13, similar to T-cell receptor alpha chain precursor V and C regions (TRA29), interferon alpha-inducible protein 27-like, RT1-CE13, MAS-related GPR member X2, and/or RT1 class lb gene H2-TL-like grc region (N3); comparing the level of the parameter of the at least one biomarker of step (d) to a control level of the parameter of the at least one biomarker, wherein the control level is associated with a lack of osteonecrosis; and characterizing the individual. The individual is characterized as being susceptible to or afflicted by osteonecrosis when the level of the parameter of the at least one biomarker of step (d) is lower than the control level of the parameter of the at least one biomarker; and lacking the susceptibility or the affliction to osteonecrosis when the level of the parameter of the at least one biomarker of step (d) is equal to or higher than the control level of the parameter of the at least one biomarker. In an embodiment, the individual has received a first dose corticosteroid therapy and the individual is characterized as being susceptible to or afflicted by osteonecrosis when the test level of the parameter (positively correlated with osteonecrosis) is higher than the control level of the parameter of the at least one biomarker after the intake of the first dose. Alternatively, the individual is characterized as lacking the susceptibility or the affliction to osteonecrosis when the test level of the parameter of the at least one biomarker (positively correlated with osteonecrosis) is equal to or lower than the control level of the parameter of the at least one biomarker after the intake of the first dose. In yet another embodiment, the individual has received a further (e.g. second) dose of corticosteroid therapy and the individual is characterized as being susceptible to or afflicted by osteonecrosis when the test level of the parameter of the at least one biomarker (positively correlated with osteonecrosis) is higher than the control level of the parameter of the at least one biomarker after the intake of the first dose and second dose. Alternatively, the individual is characterized as lacking the susceptibility or the affliction to osteonecrosis when the test level of the parameter of the at least one biomarker (positively associated with osteonecrosis) is equal to or lower than the control level of the parameter of the at least one biomarker after the intake of the second dose. In still another embodiment, the level of the parameter of the at least one biomarker is determined with an antibody. In yet another embodiment, the method can comprise managing the individual based on the characterized predisposition to osteonecrosis. In still another embodiment, the method can comprise confirming the predisposition by visualizing the bone tissue. In yet another embodiment, the biological sample is serum or blood. In still another embodiment, the individual is a human.

In a second aspect, the present invention provides a prognostic system for determining a predisposition to osteonecrosis in an individual. Broadly, the system comprises a reaction vessel for combining a biological sample from the individual and an analyte-specific reagent (ASR) for measuring a level of the parameter of at least one biomarker whose expression is positively correlated with osteonecrosis, wherein said at least one biomarker comprises alpha-2-macroglobulin (A2M); a processor in a computer system; a memory accessible by the processor; and at least one application coupled to the processor. The at least one application is configured for receiving a measure of the level of the parameter of the at least one biomarker; comparing the level of the parameter of the at least one biomarker with a control level of the parameter of the at least one biomarker, wherein the control level is associated with a lack of osteonecrosis; and characterizing the individual as being susceptible to or afflicted by osteonecrosis when the level of the parameter of the at least one biomarker is higher than the control level of the at least one biomarker and as lacking the susceptibility or the affliction to osteonecrosis when the level of the parameter of the at least one biomarker is equal to or lower than the control level of the parameter of the at least one biomarker. In an embodiment, the at least one biomarker further comprises collagen type II alpha-1 (col2A1). In another embodiment, the at least one biomarker further comprises melanoma inhibitory activity 1 (MIA1). In still a further embodiment, the at least one biomarker further comprises a steroid stimulus response gene selected from the group consisting of alkaline phosphatase, tissue-nonspecific, transforming growth factor beta 2 and potassium large conductance calcium-activated channel, subfamily m, alpha member 1. In still another embodiment, the at least biomarker further comprises an apoptosis pathway response gene selected from the group consisting of S100 protein-beta polypeptide, transforming growth factor-beta 2, vitamin D receptor, unc-5 homolog c and growth hormone receptor. In yet another embodiment, the at least one biomarker further comprises scrapie responsive gene 1, growth hormone receptor, SH2B adaptor protein 2, fibromodulin, matrix metallopeptidase 3, Proprotein convertase subtilisin/kexin type 6, cadherin 13, calpain 6, murinoglobulin 2, solute carrier family 38, member 3, fibroblast growth factor 1, vitamin D receptor, carbonic anhydrase 8, WNT1 inducible signaling pathway protein 2, integrin binding sialoprotein, calcitonin receptor, S100 protein, beta polypeptide, neural, potassium large conductance calcium-activated channel, subfamily M, alpha member 1, angiopoietin-like 2, pannexin 3, sphingomyelin phosphodiesterase 3, neutral solute carrier family 13 (sodium-dependent citrate transporter) member 5, cadherin 17, unc-5 homolog C, plasminogen activator inhibitor-1, solute carrier organic anion transporter family, member 2a1, melanoma cell adhesion molecule, orosomucoid 1, transforming growth factor beta 2, alkaline phosphatase liver/bone/kidney, basic helix-loop-helix domain containing class B3, immunoglobulin superfamily member 10, transmembrane protein 100. parathyroid hormone receptor 1, a disintegrin-like and metallopeptidase (reprolysin type) with thrombospondin type 1 motif 1, sphingomyelin synthase 2, transient receptor potential cation channel, subfamily V member 4, parvin alpha, regulator of calcineurin 2, latexin, receptor accessory protein 6 and/or cAMP responsive element binding protein 3-like 1. Optionally, the at least one application coupled to the processor is further configured for receiving a measure of a level of a parameter of at least one biomarker whose expression is negatively correlated with osteonecrosis in the biological sample from the individual, wherein said at least one biomarker is chemokine (C-X-C motif) ligand 13, similar to T-cell receptor alpha chain precursor V and C regions (TRA29), interferon alpha-inducible protein 27-like, RT1-CE13, MAS-related GPR member X2, and/or RT1 class lb gene H2-TL-like grc region (N3); comparing the level of the parameter of the at least one biomarker of step (e) to a control level of the parameter of the at least one biomarker, wherein the control level is associated with a lack of osteonecrosis; and characterizing the individual. In an embodiment, the individual has received a first dose corticosteroid therapy and the individual is characterized as being susceptible to or afflicted by osteonecrosis when the test level of the parameter (positively correlated with osteonecrosis) is higher than the control level of the parameter of the at least one biomarker after the intake of the first dose. Alternatively, the individual is characterized as lacking the susceptibility or the affliction to osteonecrosis when the test level of the parameter of the at least one biomarker (positively correlated with osteonecrosis) is equal to or lower than the control level of the parameter of the at least one biomarker after the intake of the first dose. In yet another embodiment, the individual has received a further (e.g. second) dose of corticosteroid therapy and the individual is characterized as being susceptible to or afflicted by osteonecrosis when the test level of the parameter of the at least one biomarker (positively correlated with osteonecrosis) is higher than the control level of the parameter of the at least one biomarker after the intake of the first dose and second dose. Alternatively, the individual is characterized as lacking the susceptibility or the affliction to osteonecrosis when the test level of the parameter of the at least one biomarker (positively associated with osteonecrosis) is equal to or lower than the control level of the parameter of the at least one biomarker after the intake of the second dose. In another embodiment, the level of the parameter of the at least one biomarker is determined with an antibody. In still another embodiment, the biological sample is serum or blood. In still a further embodiment, the individual is a human.

According to a third aspect, the present invention provides a software product embodied on a computer readable medium and comprising instructions for determining predisposition of an individual to osteonecrosis. It comprises a receiving module for receiving a measurement of a level of a parameter of at least one biomarker in a biological sample from the individual, wherein the expression of the at least one biomarker is positively correlated with osteonecrosis and comprises alpha-2-macroglobulin; a comparison module for comparing the level of the parameter of the at least one biomarker to a control level of the at least one biomarker, wherein the control level is associated with a lack of osteonecrosis; and (c) a characterization module for characterizing the predisposition of the individual to osteonecrosis or the presence of osteonecrosis in the individual. The individual is characterized as being susceptible to or afflicted by osteonecrosis when the level of the parameter of the at least one biomarker is higher than the control level of the at least one biomarker; and lacking the susceptibility or the affliction to osteonecrosis when the level of the parameter of the at least one biomarker is equal to or lower than the control level of the parameter of the at least one biomarker. In an embodiment, the at least one biomarker further comprises collagen type II alpha-1 (col2A1). In a further embodiment, the at least one biomarker further comprises melanoma inhibitory activity 1 (MIA1). In yet another embodiment, the at least one biomarker further comprises a steroid stimulus response gene selected from the group consisting of alkaline phosphatase, tissue-nonspecific, transforming growth factor beta 2 and potassium large conductance calcium-activated channel, subfamily m, alpha member 1. In still another embodiment, the at least biomarker further comprises an apoptosis pathway response gene selected from the group consisting of S100 protein-beta polypeptide, transforming growth factor-beta 2, vitamin D receptor, unc-5 homolog c and growth hormone receptor. In a further embodiment, the at least one biomarker further comprises scrapie responsive gene 1, growth hormone receptor, SH2B adaptor protein 2, fibromodulin, matrix metallopeptidase 3, Proprotein convertase subtilisin/kexin type 6, cadherin 13, calpain 6, murinoglobulin 2, solute carrier family 38, member 3, fibroblast growth factor 1, vitamin D receptor, carbonic anhydrase 8, WNT1 inducible signaling pathway protein 2, integrin binding sialoprotein, calcitonin receptor, S100 protein, beta polypeptide, neural, potassium large conductance calcium-activated channel, subfamily M, alpha member 1, angiopoietin-like 2, pannexin 3, sphingomyelin phosphodiesterase 3, neutral solute carrier family 13 (sodium-dependent citrate transporter) member 5, cadherin 17, unc-5 homolog C, plasminogen activator inhibitor-1, solute carrier organic anion transporter family, member 2a1, melanoma cell adhesion molecule, orosomucoid 1, transforming growth factor beta 2, alkaline phosphatase liver/bone/kidney, basic helix-loop-helix domain containing class B3, immunoglobulin superfamily member 10, transmembrane protein 100. parathyroid hormone receptor 1, a disintegrin-like and metallopeptidase (reprolysin type) with thrombospondin type 1 motif 1, sphingomyelin synthase 2, transient receptor potential cation channel, subfamily V member 4, parvin alpha, regulator of calcineurin 2, latexin, receptor accessory protein 6 and/or cAMP responsive element binding protein 3-like 1. Optionally, the software product can also include a receiving module is for receiving a measure of a level of a parameter of at least one biomarker whose expression is negatively correlated with osteonecrosis in the biological sample from the individual, wherein said at least one biomarker is chemokine (C-X-C motif) ligand 13, similar to T-cell receptor alpha chain precursor V and C regions (TRA29), interferon alpha-inducible protein 27-like, RT1-CE13, MAS-related GPR member X2, and/or RT1 class lb gene H2-TL-like grc region (N3); a comparison module is for comparing the level of the parameter of the at least one biomarker of step (d) to a control level of the parameter of the at least one biomarker, wherein the control level is associated with a lack of osteonecrosis; and a characterization module is for characterizing the individual. The individual is characterized as being susceptible to or afflicted by osteonecrosis when the level of the parameter of the at least one biomarker of step (d) is lower than the control level of the biomarker; and lacking the susceptibility or the affliction to osteonecrosis when the level of the parameter of the at least one biomarker of step (d) is equal to or higher than the control level of the parameter of the biomarker. In an embodiment, the individual has received a first dose corticosteroid therapy and the individual is characterized as being susceptible to or afflicted by osteonecrosis when the test level of the parameter (positively correlated with osteonecrosis) is higher than the control level of the parameter of the at least one biomarker after the intake of the first dose. Alternatively, the individual is characterized as lacking the susceptibility or the affliction to osteonecrosis when the test level of the parameter of the at least one biomarker (positively correlated with osteonecrosis) is equal to or lower than the control level of the parameter of the at least one biomarker after the intake of the first dose. In yet another embodiment, the individual has received a further (e.g. second) dose of corticosteroid therapy and the individual is characterized as being susceptible to or afflicted by osteonecrosis when the test level of the parameter of the at least one biomarker (positively correlated with osteonecrosis) is higher than the control level of the parameter of the at least one biomarker after the intake of the first dose and second dose. Alternatively, the individual is characterized as lacking the susceptibility or the affliction to osteonecrosis when the test level of the parameter of the at least one biomarker (positively associated with osteonecrosis) is equal to or lower than the control level of the parameter of the at least one biomarker after the intake of the second dose. In another embodiment, the level of the parameter of the at least one biomarker is determined with an antibody. In still another embodiment, the biological sample is serum or blood. In yet another embodiment, the individual is a human.

According to a fourth aspect, the present invention provides a method for determining the usefulness of an agent in the prevention of osteonecrosis. The method broadly comprises obtaining a first value of the parameter of an alpha-2-macroglobulin (A2M)-based reagent in a cell in the absence of a corticosteroid and in the presence of the agent; obtaining a second value of the parameter of an A2M-based reagent in a cell previously treating with the agent and being contacted with a corticosteroid and the agent; comparing the first value of the parameter of the A2M-based reagent with the second value of the A2M-based reagent; and characterizing the agent. The agent is characterized as being useful in the prevention of osteonecrosis when the second value of the parameter of the A2M-based reagent is equal to or lower than the first value of the A2M-based reagent; and lacking the usefulness in the prevention of osteonecrosis when the second value of the parameter of the A2M-based reagent is higher than the first value of the A2M-based reagent. In an embodiment, the A2M-based reagent is a A2M polypeptide and still, in a further embodiment, the parameter of the A2M polypeptide is the level of signaling from a low density lipoprotein receptor-related protein and/or a glucose regulator protein of 78 kDa (GRP780), the level of removal of an osteogenic growth peptide (OGP) and/or the level of inhibition of bone morphogenic protein-1 (BMP-1). In another embodiment, the A2M-based reagent is a nucleic acid molecule encoding a A2M polypeptide and still in a further embodiment, the parameter is the level of expression of the nucleic acid molecule.

According to a fifth aspect, the present invention provides a method for determining the usefulness of an agent in the treatment of osteonecrosis. Broadly the method comprises obtaining a first value of a parameter of an alpha-2-macroglobulin (A2M)-based reagent in a cell in the presence of a corticosteroid and in the absence of the agent; obtaining a second value of the parameter of the A2M-based reagent in a cell previously treated with the corticosteroid and being contacted with the agent; comparing the first value of the parameter of the A2M-based reagent with the second value of the A2M-based reagent; and characterizing the agent. The agent is characterized as being useful in the treatment of osteonecrosis when the second value of the parameter of the A2M-based reagent is equal to or lower than the first value of the A2M-based reagent; and lacking the usefulness in the treatment of osteonecrosis when the second value of the parameter of the A2M-based reagent is higher than the first value of the A2M-based reagent. In an embodiment, the A2M-based reagent is a A2M polypeptide and still, in a further embodiment, the parameter of the A2M polypeptide is the level of signaling from a low density lipoprotein receptor-related protein (LRP1) and/or a glucose regulator protein of 78 kDa (GRP78), the level of removal of an osteogenic growth peptide (OGP) and/or the level of inhibition of bone morphogenic protein-1 (BMP-1). In another embodiment, the A2M-based reagent is a nucleic acid molecule encoding a A2M polypeptide and still in a further embodiment, the parameter is the level of expression of the nucleic acid molecule.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 illustrates the histology findings in placebo and steroid-induced ON rats. (A) Photomicrographs showing histological findings in placebo-(I-IV) and steroid-treated WKY rats (V-VIII & IX-XII) femoral heads. I-IV: no osteonecrosis, normal osteocytes (arrow), V-VIII: early stage of osteonecrosis, normal osteocytes (arrow), empty lacunae (arrow head), IX-XII: late stage of osteonecrosis, empty lacunae (arrow head), complete necrosis of bone marrow (asterisk), H&E staining, I, V, IX x20; II, VI, X x40; III, VII, XI x100; IV, VIII, XII x200, dotted square chosen to be magnified. (B) Photomicrographs showing apoptosis of osteocytes as a marker of early ON. TUNEL staining apoptosis assay counterstained with 0.5% methyl green solution. I-III: normal femoral head tissues in placebo-treated WKY rats, normal osteocytes (arrow head), normal bone marrow (double asterisk), IV-VI: early stage of osteonecrosis in steroid-treated WKY rats, TUNEL positive osteocytes (arrow), empty lacunae (dotted arrow), normal osteocytes (arrow head), normal bone marrow (double asterisk), VII-IX: late stage of osteonecrosis in steroid-treated WKY rats, TUNEL positive osteocytes (arrow), empty lacunae (dotted arrow), complete necrotic bone marrow (asterisk), I, IV, VII x40; II, V, VIII x100; III, VI, IX x200, dotted square chosen to be magnified.

FIG. 2 illustrates the upregulation of A2M surface protein expression in steroid-induced early ON. Immunohistochemistry comparing the A2M (A, B), COL2A1 (C, D) and MIA (CD RAP) (E, F) protein expression between G1 (A, C, E) and G2 (B, D, F) WKY rats, showing enhancement of A2M expression in G2 compared to G1 but no enhancement shown for COL2A1 and MIA genes; brown color demonstrates protein expression and green color displays intact nucleus of cells, A-D x40 E, F x100.

FIG. 3 illustrates the Alpha-2-Macroglobulin (A2M) protein level in the blood of cortisone-treated rats. Mean concentration of blood A2M level (mg/L) is provided in function of time (months) for Fisher rats treated with placebo (A for AVN(+) (▪) and AVN(−) (▴) animals), WK rats treated with placebo (B for AVN(+) (▪) and AVN(−) (▴) animals), steroid-treated AVN(+) Fisher rats (C) and steroid-treated AVN(+) WK rats (D). Panel (E) is a combination of both panels (C, Fisher rats represented by ▴ and the broken line) and (D, WK rats represented by ♦ and the solid line).

FIG. 4 illustrates A2M gene expression levels in various treated HUVEC cells. Results are provided as relative expression levels of A2M mRNA for various HUVECs. (A) shows a dose response curve of HUVEC cells treated 48h using different concentration of dexamethasone (HUVEC-CTRL: OmM, HUVEC-DEX 0.1:0.1 mM, HUVEC-DEX-0.5:0.5 mM, HUVEC-DEX-0.75:0.75 mM, HUVEC-DEX-1:1.0 mM). (B) shows a time response of HUVEC cells treated with 1.0 mM dexamethasone at different time periods (CTRL: 0h, DEX 4h: 4h, DEX 8h: 8h, DEX 16h: 16h, DEX 24h: 24h). (C) shows response to thrombin, whitaferin (a NFκB inhibitor) and dexamethasone (CTRL: control, HUVEC-THR: thrombin treatment, HUVEC-DEX: dexamethasone treatment, HUVEC-DEX+THR: combined thrombin and dexamethasone treatment, HUVEC-DEX+WHI: combined dexamethasone and whitaferin treatment, HUVEC-DEX+WHI+THR: combined thrombin, whitaferin and thrombin treatment). (D) shows response to whitaferin and PD (a MAPK inhibitor) (CTRL; control, DEX: dexamethasone treatment, DEX+PD: combined dexamethasone and PD treatment, DEX+WA: combined dexamethasone and whitaferin treatment, WA: whitaferin treatment, PD: PD treatment).

FIG. 5 shows representative micro-computerized tomography scanning of femoral head cross-sections of control animals and dexamethasone-treated Fisher rats (A) and dexamethasone-treated WK rats (B).

### DETAILED DESCRIPTION

Alpha-2-Macroglobulin (A2M).

This polypeptide is a plasma-derived matrix metalloproteinase inhibitor which obstructs cartilage degradation induced by matrix metalloproteinases. A2M has been shown to bind to and remove the osteogenic growth peptide (OGP) from the bone system. A2M has also been show to inhibit the activity of BMP-1 (bone morphogenic protein-1). A2M has been identified on the luminal surface of endothelial cells in sections of normal human arteries and veins. A2M has been implicated in hemostasis as a regulator of thrombin activity. A2M has been also implied in the development of thromboembolism in children. Thus, A2M shares haemostatic, cartilaginous and osteogenic properties. As shown herein, A2M expression is positively correlated with osteonecrosis onset showing its importance in the pathogenesis of osteonecrosis and its use as a biomarker for detecting osteonecrosis.

Alpha-2-Macroglobulin (A2M)-Based Reagent.

In the methods and assays described herein, an A2M-based reagent will be used to determine the usefulness of an agent to prevent and/or treat osteonecrosis. Such A2M-based reagents include, but are not limited to A2M-encoding polynucleotides and A2M polypeptides.

Alpha-2-Macroglobulin (A2M)-Encoding Polynucleotides.

In the screening assays provided herewith, a full length nucleotide sequence encoding the A2M polypeptide or a fragment thereof can be used. A “fragment” of a A2M-encoding nucleotide sequence that encodes a biologically active portion (e.g. that retains A2M specific inhibition of bone remodeling activity) of A2M protein will encode at least 15, 25, 30, 50, 75, 100, 125, 150, 175, 200, 225, 250, 275, 300, 325, 500, 600, 700, 800, 900, 1 000, 1 100, 1 200, 1 300 or 1 400 contiguous amino acids, or up to the total number of amino acids present in a full-length A2M polypeptide. Fragments of the A2M-encoding nucleotide sequence that are useful as specific hybridization probes and/or as specific PCR primers generally need not encode a biologically active portion of the A2M polypeptide. Some full-length A2M-encoding polynucleotides include those have Accession Number NG_{—}011717.1 (gene), NM_{—}000014.4 9 (mRNA) and FWP007 (ORF name of cDNA).

Nucleic acid molecules that are variants of the A2M-encoding nucleotide sequences disclosed herein can also be used. “Variants” of A2M nucleotide sequences include those sequences that encode A2M proteins disclosed herein but that differ conservatively because of the degeneracy of the genetic code. These naturally occurring allelic variants can be identified with the use of well-known molecular biology techniques, such as polymerase chain reaction (PCR) and hybridization techniques. Variant nucleotide sequences also include synthetically derived nucleotide sequences that have been generated, for example, by using site-directed mutagenesis but which still encode the A2M proteins. Generally, nucleotide sequence variants of the invention will have at least 45%, 55%, 65%, 75%, 85%, 95%, or 98% identity to a particular nucleotide sequence disclosed herein. A variant A2M-encoding nucleotide sequence will encode an A2M protein that has an amino acid sequence having at least 45%, 55%, 65%, 75%, 85%, 95%, or 98% identity to the amino acid sequence of A2M protein disclosed herein. It will be appreciated by those skilled in the art that DNA sequence polymorphisms that lead to changes in the amino acid sequences of A2M proteins may exist within a population (e.g., the human population). Such genetic polymorphism in A2M gene may exist among individuals within a population due to natural allelic variation. Any and all such nucleotide variations and resulting amino acid polymorphisms or variations in A2M sequence that are the result of natural allelic variation and that do not alter the functional activity of A2M proteins are intended to be used herein.

In addition to naturally-occurring allelic variants of A2M-encoding sequences that may exist in the population, the skilled artisan will further appreciate that changes can be introduced by mutation into the nucleotide sequences of the invention thereby leading to changes in the amino acid sequence of the encoded A2M proteins, without altering the biological activity of the A2M proteins. Such mutations can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed. A “conservative amino acid substitution” is one in which the amino acid residue is replaced with an amino acid residue having a similar side chain. Families of amino acid residues having similar side chains have been defined in the art. These families include amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g. alanine, valine, leucine, isoleucine, proline, phenylalanine, methionine, tryptophan), beta-branched side chains (e.g. threonine, valine, isoleucine) and aromatic side chains (e.g. tyrosine, phenylalanine, tryptophan, histidine).

In the some of the screening assays provided herewith, it is also possible to use the promoter of an A2M-encoding nucleic acid to determine if a specific agent is capable of inhibiting or lowering A2M expression. In some assay format, it may be advisable to operably link the A2M promoter to a reporter gene. The reporter gene can encode a protein that can be detected in the reaction vessel. The reporter gene can be, for example, the A2M gene itself or any other gene encoding a protein that can be detected in the reaction vessel (for example a fluorescent protein or a β-galactosidase protein).

Alpha-2-Macroglobulin Polypeptide and Related Products.

The A2M-based reagent maybe the full-length A2M polypeptide or a biologically active fragment of the A2M polypeptide that retains its characteristic lipase activity. “Fragments” or “biologically active portions” of the A2M polypeptide include polypeptide fragments comprising amino acid sequences sufficiently identical to or derived from the amino acid sequence of the A2M polypeptide and exhibiting at least one activity of the A2M polypeptide (such as A2M-specific inhibition of bone remodeling activity), but which include fewer amino acids than the full-length A2M polypeptide. Typically, biologically active portions comprise a domain or motif with at least one activity of the A2M polypeptide. A biologically active portion of the A2M polypeptide can be a polypeptide that is, for example, 10, 25, 50, 100, 150, 200, 250, 300, 400, 500, 600, 700, 800, 900, 1 000, 1 100, 1 200, 1 300, 1 400 or more amino acids in length. Such biologically active portions can be prepared by recombinant techniques and evaluated for one or more of the functional activities of a native A2M polypeptide. A exemplary A2M-polypeptide is the one having Accession Number NP_{—}000005.2. Variants of such peptides do exist and one of them is presented under Accession Number NP_{—}000005.2. Two shorter forms of A2M have been published and possess 353 and 643 amino acids, respectively.

Analyte-Specific Reagent (ASR).

The term “analyte specific reagent” or “ASR” refers to any molecule including any chemical, nucleic acid sequence, polypeptide (e.g. receptor protein) or composite molecule and/or any composition that permits quantitative assessment of the biomarker level. For example, the ASR can be for example a nucleic acid probe primer set, comprising a detectable label or aptamer that binds to, reacts with and/or responds to the A2M gene or its related sequences (mRNA, cDNA, etc.). A gene-specific ASR is herein referred to by reference to the gene, for example a “A2M ASR” refers to an ASR such as a probe that specifically binds to a A2M gene product in a manner to permit quantification of the A2M gene product (e.g. mRNA corresponding of cDNA). A polypeptide-specific ASR is herein referred to by reference to the polypeptide or protein, for example a “A2M ASR” refers to an ASR such as an antibody that specifically binds to the A2M protein in a manner to permit quantification of the A2M protein. The term “specifically binds” as used herein refers to a binding reaction that is determinative of the presence of the analyte (e.g. polypeptide or nucleic acid) often in a heterogeneous population of macromolecules. For example, when the ASR is a probe, it specifically binds refers to the specified target under hybridization conditions binds to a particular gene sequence at least 1.5, at least 2 or at least 3 times background.

Antibody.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, diabodies, bispecific antibody fragments and other fragments can also be synthesized by recombinant techniques.

Biological Sample.

As used herein, a biological sample refers to sample from the individual suspected of being predisposed to or afflicted by osteonecrosis. This sample can be a bodily fluid (such as serum, plasma, whole blood, saliva or urine) or a tissue sample (such as bone +/− cartilage, extracted from the affected site). In an embodiment, the biological sample can comprise a cell (such as an endothelial cell).

Correlation with Osteonecrosis.

The expression of the biomarkers presented herewith and used in the different methods is either upregulated or downregulated with the predisposition or onset of osteonecrosis. The expression of a biomarker is said to be positively correlated with osteonecrosis, when it is upregulated with the predisposition or onset of osteonecrosis. The expression of a biomarker is said to be negatively correlated with osteonecrosis, when it is downregulated with the predisposition or onset of osteonecrosis.

Osteonecrosis.

As used herein, osteonecrosis is a condition observed when local blood flow is reduced, particularly in the head of the femur (hip) or other joints. Such reduced blood flow results in bone death (osteonecrosis). The exact physiopathology of this condition is not known. This devastating musculoskeletal condition can affect relatively young individuals who will need frequent joint replacement. This condition is often irreversible because it is diagnosed too late for current therapy to be efficient. At the very early stages of the disease (stage 0-2), minimal changes in the bone physiology are observed and these changes can be reversible. As the condition progresses, a typical “crescent sign” is observed (stage 3) and at this stage, the condition is irreversible. In the late stages of the condition, joint space progressively narrows (stages 4 and 5) and the acetabulum becomes involved (stage 6). Current pharmacological therapies typically include anticoagulants, such as coumadin or low molecular weight heparin. It has even been suggested that biphosphonates and prostacyclin may be used to treat the afflicted patients. And even though there is no standard medical therapy for this condition, surgery may be beneficial for replacing the afflicted bone tissue or joint. Risk factors for this condition include, but are not limited to, trauma, alcohol usage and glucocorticoid usage.

Glucocorticoid usage is becoming more and more prevalent in specific populations such as HIV-infected, cancer patients (especially acute lymphoblastic leukemia patients), stem cell recipients as well as systemic lupus erythematosus patients. The glucocorticoid market is increasing in other patient populations such as asthma, COPD (Chronic Obstructive Pulmonary Disease), rheumatoid arthritis, inflammatory bowel disease (e.g. Crohn's, ulcerative colitis), solid organ transplantation, ARDS (Acute Respiratory Distress Syndrome), nephropathies, allergic reactions, skin manifestations, vasculitis, gout, polymyalgia rheumatica (PMR), Churg-Strauss syndrome, ocular manifestations (e.g. uveitis), sarcoidosis, etc. As indicated above, the administration of glucocorticoids increases the risk of the treated individual to develop osteonecrosis. There is thus a need in these patient populations to assess the risk of the onset of osteonecrosis.

Prevention, Treatment and Alleviation of Symptoms.

These expressions refer to the ability of an agent to limit the development, progression and/or symptomatology of osteonecrosis. Symptoms associated with osteonecrosis include, but are not limited to, bone degradation, bone death, bone joint narrowing. Some of these symptoms at some of the disease stages can be visualized with X-ray. Other symptoms that the afflicted patient may feel also include pain (especially in the groin), limited range of motion of the affected joint, limping, difficulty in walking and climbing stairs.

Primer.

The term “primer” or “oligonucleotide primer” as used herein refers to a nucleic acid sequence, whether occurring naturally as in a purified restriction digest or produced synthetically, which is capable of acting as a point of synthesis of when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15 to 25 or more nucleotides, although it can contain less. The factors involved in determining the appropriate length of primer are readily known to one of ordinary skill in the art.

Probe.

The term “probe” as used herein refers to a nucleic acid sequence that comprises a sequence of nucleotides that will hybridize specifically to a target nucleic acid sequence e.g. a nucleic acid encoding the biomarkers disclosed herein. For example the probe comprises at least 10 or more bases or nucleotides that are complementary and hybridize contiguous bases and/or nucleotides in the target nucleic acid sequence. The length of probe depends on the hybridization conditions and the sequences of the probe and nucleic acid target sequence and can for example be 10 to 20, 21 to 70, 71 to 100, 101 to 500 or more bases or nucleotides in length. The probes can optionally be fixed to a solid support such as an array chip or a microarray chip.

### Methods and Systems for the Assessment of Osteonecrosis Disease Status.

A2M is particularly useful marker of steroid-induced osteonecrosis. A2M expression is modulated in the early stages of the disease, even when very little symptoms are observed. As such A2M can be successfully used for the assessment of the early stages of the disease, when medical remedies (such as drugs and surgery) are available to the patient to reverse (totally or in part) the condition. Further, this marker can also be used in pre-screening stages to determine if a more costly diagnostic procedure (such as an MRI) would be useful to determine the presence or absence of the condition.

As shown herein, the expression and/or activity of A2M has been shown to be upregulated with the onset and/or progression of osteonecrosis in individuals. Consequently, in order to assess a predisposition to osteonecrosis in an individual, the methods provide determining a level of a parameter of A2M and comparing it to a control. In an embodiment, the control is associated with a lack of osteonecrosis. It may be derived from a healthy individual (or a group of healthy individuals) that is not predisposed or afflicted to osteonecrosis and, preferably, this healthy individual (or group of healthy individuals) has never received a corticosteroid dose. In this particular embodiment, if the level of the parameter of A2M is higher then the control, then the individual is characterized as being susceptible to and maybe even be afflicted by osteonecrosis. On the other hand, if the level of the parameter of A2M is equal to or lower than the control, then the individual is characterized as not being susceptible or afflicted with osteonecrosis.

In another embodiment, the method can also provide measuring if level of a parameter of A2M is higher than a control associated with a lack of osteonecrosis. In such embodiment, the control may be derived from a healthy individual (or a group of healthy individuals) that is not predisposed or afflicted to osteonecrosis and, preferably, this healthy individual (or group of healthy individuals) has never received a corticosteroid dose. In this particular embodiment, if the measurement is made then the individual is characterized as being susceptible to and maybe even be afflicted by osteonecrosis. On the other hand, if the measurement cannot be made, then the individual is characterized as not being susceptible or afflicted with osteonecrosis.

In another embodiment, the control is associated with a predisposition to or affliction by osteonecrosis. This control may be derived from an individual (or a group of individuals) that is (are) known to be predisposed to or afflicted by osteonecrosis and, preferably, this individual (or group of individuals) has received at least one dose of a corticosteroid. In this particular embodiment, if the level of the parameter of A2M is higher than or equal to then the control, then the individual is characterized as being susceptible to and maybe even be afflicted by osteonecrosis. On the other hand, if the level of the parameter of A2M is lower than the control, then the individual is characterized as not being susceptible or afflicted with osteonecrosis.

In yet another embodiment, the measuring step comprises determining if level of the parameter of A2M is equal to or higher than a control associated with a predisposition to or affliction by osteonecrosis. This control may be derived from an individual (or a group of individuals) that is (are) known to be predisposed to or afflicted by osteonecrosis and, preferably, this individual (or group of individuals) has received at least one dose of a corticosteroid. In this particular embodiment, if the measurement can be made, then the individual is characterized as being susceptible to and maybe even be afflicted by osteonecrosis. On the other hand, if the measurement cannot be made, then the individual is characterized as not being susceptible or afflicted with osteonecrosis.

In some embodiments, it is preferable to conduct the method at least twice in an individual to determine if the modulation in the level of a parameter of A2M is acute (e.g. limited in time) or sustained (e.g. is present over a certain period of time). In some individuals, a single measurement of an elevated A2M parameter is not necessarily indicative of a susceptibility to osteonecrosis or the presence of osteonecrosis. In these individuals, it is preferable to repeat the measurement of the level of A2M parameter at least a second time to determine if the elevation is sustained. In those individuals, the presence of a sustained elevated A2M parameter is indicative of a susceptibility to osteonecrosis or the presence of osteonecrosis. For example, the multiple measurements can be made during a period of 6 months, 12 months, 24 months, 36 months, 48 months or 60 months. In another example, the first measurement can be made 4 to 6 months after the first intake of steroids and the second (or further measurements) can be made at 12 months, 24 months, 36 months, 48 months or 60 months. In alternative embodiments, measurements can be made after the individual has stopped taking the steroids to determine if the modulation of the parameter of A2M is maintained. Such maintenance is indicative of susceptibility or or affliction by osteonecrosis.

In some embodiments of the diagnostic methods, the measurement(s) can be made while the individual is still receiving the corticosteroid therapy. In other embodiments, the measurement(s) can be made after the individual has stopped taking the corticosteroid therapy.

Various parameters of A2M can be assessed to make the characterization presented herein with respect to susceptibility/affliction to osteonecrosis. One of the parameter that can be measured is the level of expression and/or stability of the A2M-encoding nucleic acid (full-length, fragments and/or variants). The level of expression and the stability of a nucleic acid-encoding A2M (such as an mRNA) are positively correlated with the disease (e.g. increased expression and/or stability of the nucleic acid-encoding A2M is associated with increased likelihood of onset of the disease). When the measurement is made at the nucleic acid level, the level of activity of the A2M promoter, the level of expression of the A2M-encoding mRNA and/or the level of stability of the A2M-encoding mRNA can be measured with techniques known in the art. In an embodiment, a specific quantifier associated (preferably associated with a detectable signal) can be used to measure the level of expression of the A2M-encoding mRNA (e.g. a nucleic acid probe linked to a detectable signal).

Another parameter that can be used is the level of expression of the A2M polypeptide. The A2M polypeptide is expressed by various types of cells. In addition, the A2M polypeptide can be detected in the serum of individuals. The level of expression of the A2M polypeptide is positively correlated with osteonecrosis (e.g. increased expression is associated with increased likelihood of onset of osteonecrosis). When the measurement is made at the polypeptide level, various methods known in the art, for example antibody-based detection or other spectrometry analyses, can be successfully used. In an embodiment, a quantifying or analyte-specific reagent, such as an antibody for example can be used. The quantifier can be associated with a detectable signal for providing the measurement.

A further parameter than can be used is the level of activity of the A2M polypeptide. As discussed herein, A2M possesses inhibition properties with respect to bone-remodeling, e.g. its biological activity limits the degradation of bone and/or cartilage. The level of activity of the A2M polypeptide is positively correlated with osteonecrosis (e.g. increased biological activity is associated with increased likelihood of onset of osteonecrosis). The biological activity that can be measured include, but are not limited to matrix metalloproteinase inhibition, binding and/or removal of the osteogenic growth peptide (OGP) from the bone system, BMP-1 (bone morphogenic protein-1) inhibition and/or regulation of thrombin activity. Another biological activity that can be monitored is the ability of A2M to bind and induce signaling from its cognate receptors (low density lipoprotein receptor-related protein and glucose regulator protein of 78 kDa). In order to provide these measurements, a quantifier can be used. Such quantifier is, in an embodiment, modified by the biological activity of A2M. The quantifier can be associated with a detectable signal for providing the measurement.

Additional markers can also be used to make the assessment. The expression of some of these markers, like A2M, are positively correlated with osteonecrosis. One of the biomarker whose expression is positively correlated with osteonecrosis is collagen type II alpha-1 (col2A1). In an embodiment, the col2A1 biomarker is used in combination with A2M and no additional biomarkers is used to make the disease assessment. In another embodiment, the col2A1 biomarker is used in combination with A2M and MIA1 and no additional biomarkers is used to make the disease assessment. In still another embodiment, the col2A1 biomarker is used in combination with other biomarker whose expression are positively and/or negatively correlated with osteonecrosis. Some polynucleotides for col2A1 are presented at Accession Number NG_{—}008072.1 (Gene), NM_{—}001844.4 (mRNA) and CCDS41778.1 (cDNA). A example of a col2A1 polypeptide is shown at Accession Number NP_{—}001835.3. Polypeptide variant have also been reported.

Another biomarker whose expression is positively correlated with osteonecrosis is the melanoma inhibitory activity 1 (MIA1) polypeptide. In an embodiment, the MIA1 biomarker is used in combination with A2M and no additional biomarkers is used to make the disease assessment. In another embodiment, the MIA1 biomarker is used in combination with A2M and col2A1 and no additional biomarkers is used to make the disease assessment. In still another embodiment, the MIA1 biomarker is used in combination with other biomarker whose expression are positively and/or negatively correlated with osteonecrosis. Some polynucleotide encoding MIA1 are presented at Accession Number NM_{—}101202553.1 (mRNA) and CCDS12566.1 (cDNA). An example of a MIA1 polypeptide is shown at Accession Number NP_{—}001189482.1.

It the present method, it is also contemplated that the assessment be made by including a biomarker considered to be a steroid stimulus response gene. These genes include, but are not limited to, alkaline phosphatase, tissue-nonspecific, transforming growth factor beta 2 and/or potassium large conductance calcium-activated channel, subfamily m, alpha member 1. It is also contemplated that the biomarker further comprises an apoptosis pathway response gene. Such gene include, but are not limited to S100 protein-beta polypeptide, transforming growth factor-beta 2, vitamin D receptor, unc-5 homolog c and/or growth hormone receptor.

Additional biomarkers whose expression is positively correlated with osteonecrosis are also provided herewith. These biomarkers include the growth hormone receptor, SH2B adaptor protein 2, fibromodulin, matrix metallopeptidase 3, Proprotein convertase subtilisin/kexin type 6, cadherin 13, calpain 6, murinoglobulin 2, solute carrier family 38, member 3, fibroblast growth factor 1, vitamin D receptor, carbonic anhydrase 8, WNT1 inducible signaling pathway protein 2, integrin binding sialoprotein, calcitonin receptor, S100 protein, beta polypeptide, neural, potassium large conductance calcium-activated channel, subfamily M, alpha member 1, angiopoietin-like 2, pannexin 3, sphingomyelin phosphodiesterase 3, neutral solute carrier family 13 (sodium-dependent citrate transporter) member 5, cadherin 17, unc-5 homolog C, plasminogen activator inhibitor-1, solute carrier organic anion transporter family, member 2a1, melanoma cell adhesion molecule, orosomucoid 1, transforming growth factor beta 2, alkaline phosphatase liver/bone/kidney, basic helix-loop-helix domain containing class B3, immunoglobulin superfamily member 10, transmembrane protein 100. parathyroid hormone receptor 1, a disintegrin-like and metallopeptidase (reprolysin type) with thrombospondin type 1 motif 1, sphingomyelin synthase 2, transient receptor potential cation channel, subfamily V member 4, parvin alpha, regulator of calcineurin 2, latexin, receptor accessory protein 6 and/or cAMP responsive element binding protein 3-like 1. In an embodiment, a subset of any one of three of these biomarkers is used in combination with A2M (optionally in combination with col2A1 and/or MIA1) to provide a disease assessment. In a further embodiment, a subset of any one of five of these biomarkers is used in combination with A2M (optionally in combination with col2A1 and/or MIA1) to provide a disease assessment. In yet another embodiment, a subset of any one of seven of these biomarkers is used in combination with A2M (optionally in combination with col2A1 and/or MIA1) to provide disease assessment. In still another embodiment, a subset of any one of ten of these biomarkers is used in combination with A2M (optionally in combination with col2A1 and/or MIA1) to provide disease assessment.

Additional biomarkers whose expression is negatively correlated with osteonecrosis are also provided herewith. These biomarkers include chemokine (C-X-C motif) ligand 13, similar to T-cell receptor alpha chain precursor V and C regions (TRA29), interferon alpha-inducible protein 27-like, RT1-CE13, MAS-related GPR member X2, and/or RT1 class lb gene H2-TL-like grc region (N3). When these biomarkers are used, they are compared to a control level (associated with a lack of osteonecrosis) to determine if the individual is susceptible to or afflicted by osteonecrosis (when the level of the parameter of the biomarker is lower than the control level) or lacks the susceptibility or the affliction to osteonecrosis (when the level of the parameter of the biomarker is equal to or higher than the control level). In an embodiment, a subset of any one of two of these biomarkers is used in combination with A2M (optionally in combination with col2A1 and/or MIA1) to provide a disease assessment. In a further embodiment, a subset of any one of four of these biomarkers is used in combination with A2M (optionally in combination with col2A1 and/or MIA1) to provide a disease assessment. In yet another embodiment, a subset of any one of six of these biomarkers is used in combination with A2M (optionally in combination with col2A1 and/or MIA1) to provide disease assessment.

The method presented herein are useful for predicting the predisposition to osteonecrosis or diagnose the onset of osteonecrosis. As indicated above, osteonecrosis can occur following corticosteroid usage. For example, osteonecrosis has been reported in individuals following a single high dose such as solumedrol (500 mg intravenous) or the chronic use of prednisone at (0.5 to 1.0 mg/kg/day orally). As such, the individuals that are submitted to the method presented herewith have received at least a dose of corticosteroid (such as a glucocorticosteroid). In an embodiment, the individual that is being assessed is under active corticosteroid therapy and the method is used to determine if the corticosteroid therapy can be continued without increasing the predisposition to osteonecrosis.

Once the predisposition to or affliction by osteonecrosis has been assessed, the individual can be managed based on the results obtained. For example, an increased predisposition to osteonecrosis or the onset of osteonecrosis has been detected which may indicate the need to recommend to the patient a non-weight bearing approach as a first step and/or a surgical application such as core decompression. If the disease is diagnosed in the early stages (I or II), it may also be beneficial to consider modifying or even discontinuing corticosteroid usage in the individual when clinically possible.

The method described herein can also be used to determine if other more costly diagnostic methods (such as imaging techniques, MRI for example) would be beneficial to determine the presence or absence of osteonecrosis in the individual. For example, if an individual is considered to lack the predisposition to osteonecrosis based on the method described herein, it indicates that no further diagnostic methods are necessary to assess osteonecrosis status. On the other hand, if an individual is considered to be at risk of developing osteonecrosis based on the method described herewith, it indicates that other diagnostic means, such as an MRI, may be beneficial to confirm the presence of the disease or the stage of the disease. In addition, the additional diagnostic means can also be used to determined if corticosteroid therapy can be continued (if no signs of the disease can be detected) or should be modified (if symptoms of the disease can be detected).

The present application also provides a prognostic system to carry on the method described herewith. This prognostic system comprises a reaction vessel, a processor in a computer system, a memory accessible by the processor and at least one application coupled to the processor. The reaction vessel is intended to combine the biological sample from the individual and an analyte-specific reagent (ASR) for measuring a level of the parameter of at least one biomarker whose expression is positively correlated with osteonecrosis. The at least one application is configured for receiving a measurement of the biomarker level, comparing it to a control level and characterizing the individual susceptibility to/affliction by osteonecrosis based on the comparison.

The present application also provides a software product embodied on a computer readable medium and comprising instructions for determining predisposition of an individual to osteonecrosis. The product comprises the following: a receiving module (for receiving a measurement of a level of a parameter of the biomarker), a comparison module (for comparing the level of the parameter of the biomarker to a control level); and a characterization module for characterizing the predisposition of the individual to osteonecrosis based on the comparison.

### Methods and Systems for the Characterization of a Usefulness of an Agent as a Preventive Therapy for Osteonecrosis

As shown herein, the level of expression of A2M is upregulated in the very first steps of osteonecrosis even when no symptoms are experienced by the afflicted individual and, in some embodiments, even when no histopathological symptoms are observed. Consequently, because the level of expression of A2M is associated with the onset of osteonecrosis, the level of expression of A2M can be used to screen for potential agents for preventing the onset of osteonecrosis. Agents that fail to increase A2M expression level or activity in the presence of corticosteroid will be considered useful as a preventive measure for osteonecrosis.

In the methods and systems described for screening agents useful in the prevention of osteonecrosis, the A2M-based reagent is mixed first with the agent to be screened and then with a corticosteroid. The level of the parameter of the A2M-based reagent is assessed prior to and after contact with the corticosteroid. These two levels are then compared to determine the usefulness of the agent to prevent osteonecrosis. If the presence of the agent prevents the upregulation of the expression of the A2M-based reagent upon the addition of the corticosteroid, then it is determined that the agent is useful for the prevention of osteonecrosis.

In a first step, an agent to be screened is combined in a reaction vessel with an A2M-based reagent. The reaction vessel is designed as such that, upon the addition of a corticosteroid, the level of the parameter of the A2M-based reagent (in the absence of the agent) would be upregulated. For screening applications, a suitable in vitro environment for the screening assay described herewith can be a cultured cell. Such cell should be able to maintain viability in culture. The cultured cell(s) should (i) express a polynucleotide encoding A2M or biologically active variant thereof (ii) express a A2M-encoding polynucleotide or variant thereof or related chimeric protein and/or (iii) comprise the A2M promoter region. If a primary cell culture is used, the cell may be isolated (like a HUVEC) or in a tissue-like structure, for example, as part of a bone-like structure. A further suitable environment is a non-human model, such as an animal model. If the characterization of the agent occurs in a non-human model, then the model (such as a rodent) is administered with the agent. Various dosage and modes of administration maybe used to fully characterize the agent's ability to prevent osteonecrosis.

Once the agent has been combined in the reaction vessel with the A2M-based reagent, a measurement or value of a parameter of the A2M-based reagent is made. This assessment may be made directly in the reaction vessel (by using a probe) or on a sample of such reaction vessel. The measurement of the parameter of the A2M-based reagent can be made either at the DNA level, the RNA level and/or the polypeptide level.

The measuring step can rely on the addition of a quantifier specific to the parameter to be assessed to the reaction vessel or a sample thereof. The quantifier can specifically bind to a parameter of a A2M-based reagent that is being assessed, such as, for example, a nucleotide product encoding A2M or a A2M polypeptide. In those instances, the amount of the quantifier that specifically bound (or that did not bind) to the A2M-based reagent can be determined to provide a measurement of the parameter of the A2M-based reagent. In another embodiment, the quantifier can be modified by a parameter of the A2M-based reagent. In this specific instance, the amount of modified (or unmodified) quantifier will be determined to provide a measurement of the parameter of the A2M-based reagent. In an embodiment, the signal of the quantifier can be provided by a label that is either directly or indirectly linked to a quantifier.

Various parameters of the A2M-based reagent can be measured. For example, when the A2M-based reagent is a A2M polypeptide or fragment thereof, the parameter that is measured can be the polypeptide biological activity (ability to bind to its cognate receptors and elicit cellular signaling, removal of OGP or inhibition of BMP-1), the polypeptide quantity and/or stability. When the A2M-based reagent is a nucleotide encoding a A2M polypeptide or fragment thereof, the parameter can be the level of expression or stability of the A2M-encoding nucleotide. Even though a single parameter is required to enable the characterization of the agent, it is also provided that more than one parameter of the A2M-based reagent may be measured.

If the measurement of the parameter is performed at the nucleotide level, then the transcription activity of the promoter associated with the A2M gene can be assessed. This assessment can be made, for example, by placing a reporter vector (such as a luciferase reporter based assay) in the presence of the A2M polypeptide (inside or outside a cell). Such reporter vectors can include, but are not limited to, the promoter region of the A2M gene (or fragment thereof) operably linked to a nucleotide encoding a reporter polypeptide (such as, for example, A2M, β-galactosidase, green-fluorescent protein, yellow-fluorescent protein, etc.). Upon the addition of the biological sample or the agent, the promotion of transcription from the promoter of the A2M gene is measured indirectly by measuring the transcription of the reporter polypeptide. In this particular embodiment, the quantifier is the reporter polypeptide and the signal associated to this quantifier that is being measured will vary upon the reporter polypeptide used. Alternatively or complementarily, the stability and/or the expression level of the A2M-encoding nucleotide can be assessed by quantifying the amount of a A2M-encoding nucleotide (for example using qPCR) or the stability of such nucleotide.

In one screening assay format, the expression of a nucleic acid encoding A2M in a cell or tissue sample is monitored directly by hybridization to the nucleic acids specific for A2M. In another assay format, cell lines or tissues can be exposed to the agent to be tested under appropriate conditions and time, and total RNA or mRNA isolated, optionally amplified, and quantified.

If the measurement of the parameter is performed at the polypeptide level, an assessment of A2M biological activity can be performed. A2M is a transcription factor that removes OGP, downregulates and/or inhibits BMP-1. As such, one of A2M's biological activity is to bind to other transcription regulators (also referred to as binding partners) as well as to bind to its target sequences.

Such evaluation can be made in vitro. The reaction mixture can include a binding partner or potentially interacting fragment thereof. This type of assay can be accomplished, for example, by coupling one of the components, with a label such that binding of the labeled component to the other can be determined by detecting the either directly or indirectly, and the radioisotope detected by direct counting of radioemission or by scintillation counting. Alternatively, a component can be enzymatically labeled with, for example, horseradish peroxidase, alkaline phosphatase, or luciferase, and the enzymatic label detected by determination of conversion of an appropriate substrate to product. Competition assays can also be used to evaluate a physical interaction between a test compound and a target.

In another assay format, A2M's activity can be indirectly measured by determining its ability to bind to its cognate receptor and elicit signaling from these receptors. A2M binds to at least two different cell surface receptors. The first A2M cell surface receptor is the low density lipoprotein receptor-related protein 1 (LRP1), involved in a broad range of proteins and is involved in many biological processes and diseases. The second A2M cell surface protein is glucose regulated protein of 78 kDa (GRP78) expressed in the ER, and plays an important role for the folding and maturation of proteins.

Cell-free screening assays usually involve preparing a reaction mixture of the target protein and the test compound under conditions and for a time sufficient to allow the two components to interact and bind, thus forming a complex that can be removed and/or detected.

The interaction between two molecules can also be detected, e.g., using a fluorescence assay in which at least one molecule is fluorescently labeled. One example of such an assay includes fluorescence energy transfer (FET or FRET for fluorescence resonance energy transfer). A fluorophore label on the first “donor” molecule is selected such that its emitted fluorescent energy will be absorbed by a fluorescent label on a second “acceptor” molecule, which in turn is able to fluoresce due to the absorbed energy. Alternately, the “donor” protein molecule may simply utilize the natural fluorescent energy of tryptophan residues. Labels are chosen that emit different wavelengths of light, such that the “acceptor” molecule label may be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. A FET binding event can be conveniently measured through standard fluorometric detection means well known in the art (e.g., using a fluorometer).

Another example of a fluorescence assay is fluorescence polarization (FP). For FP, only one component needs to be labeled. A binding interaction is detected by a change in molecular size of the labeled component. The size change alters the tumbling rate of the component in solution and is detected as a change in FP.

In another embodiment, the measuring step can rely on the use of real-time Biomolecular Interaction Analysis (BIA). “Surface plasmon resonance” or “BIA” detects biospecific interactions in real time, without labeling any of the interactants (e.g. BlAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the A2M-reagent is anchored onto a solid phase. The A2M-based reagent-related complexes anchored on the solid phase can be detected at the end of the reaction, e.g. the binding reaction. For example, the A2M-based reagent can be anchored onto a solid surface, and the test compound, (which is not anchored), can be labeled, either directly or indirectly, with detectable labels discussed herein. Examples of such solid phase include microtiter plates, test tubes, array slides, beads and micro-centrifuge tubes. In one embodiment, a A2M chimeric protein can be provided which adds a domain that allows one or both of the proteins to be bound to a matrix. Following incubation, the vessels are washed to remove any unbound components, the matrix immobilized in the case of beads, complex determined either directly or indirectly, for example, as described above. Alternatively, the complexes can be dissociated from the matrix, and the level of A2M binding or activity determined using standard techniques.

In order to conduct the assay, the non-immobilized component (agent or biological agent) is added to the coated surface containing the anchored component. After the reaction is complete, unreacted components are removed (e.g. by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface, e.g., using a labeled antibody specific for the immobilized component (the antibody, in turn, can be directly labeled or indirectly labeled with, e.g. a labeled anti-Ig antibody).

In one embodiment, this assay is performed utilizing antibodies specific to A2M or target molecules but which do not interfere with binding of the A2M to its target molecule. Such antibodies can be derivatized to the surface, and unbound target or the A2M-based reagent trapped on the surface by antibody conjugation. Methods for detecting such complexes, in addition to those described above for the GST-immobilized complexes, include immunodetection of complexes using antibodies reactive with the A2M-based reagent or target molecule, as well as enzyme-linked assays which rely on detecting an enzymatic activity associated with the A2M-based reagent or target molecule.

Alternatively, cell free assays can be conducted in a liquid phase. In such an assay, the reaction products are separated from unreacted components, by any of a number of standard techniques, including but not limited to: differential centrifugation; chromatography (gel filtration chromatography, ion-exchange chromatography) and/or electrophoresis. Such resins and chromatographic techniques are known to one skilled in the art. Further, fluorescence energy transfer may also be conveniently utilized, as described herein, to detect binding without further purification of the complex from solution.

To identify agents that facilitate with the interaction between the target product and its binding partner(s), for example, a reaction mixture containing the A2M-based reagent and the binding partner is prepared, under conditions and for a time sufficient, to allow the two products to form complex. In order to test if an agent which facilitates the interaction between A2M and its binding partner, the reaction mixture can be provided in the presence and absence of the test agent. The test agent can be initially included in the reaction mixture, or can be added at a time subsequent to the addition of the target and its cellular or extracellular binding partner. Control reaction mixtures are incubated without the test agent or with vehicle. The formation of any complexes between the target product and the cellular or extracellular binding partner is then detected. The formation of a complex in the reaction mixture containing the test compound, but not in the control reaction, indicates that the test agent facilitates the interaction of the A2M-based reagent and the interactive binding partner. In an embodiment, it is possible to detect the formation of the A2M-based complex indirectly by measuring the level of expression of a reporter gene whose expression is modulated by the presence (or absence) of the complex.

These assays can be conducted in a heterogeneous or homogeneous format. Heterogeneous assays involve anchoring either the A2M-based reagent or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the agents being tested. For example, test agents that interfere with the interaction between the A2M-based reagent and the binding partners, e.g., by competition, can be identified by conducting the reaction in the presence of the test substance. Alternatively, test agents that facilitate preformed complexes, can be tested by adding the test compound to the reaction mixture prior to complexes have been formed. The various formats are briefly described below.

In a heterogeneous assay system, either the A2M-based reagent or the binding partner, is anchored onto a solid surface (e.g. a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the agent. After the reaction is complete, unreacted components are removed (e.g. by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g. using a labeled antibody specific for the initially non-immobilized species (the antibody, in turn, can be directly labeled or indirectly labeled with, e.g. a labeled anti-Ig antibody). Depending upon the order of addition of reaction components, agents that enable complex formation or that promote the stability of preformed complexes can be detected.

Alternatively, the reaction can be conducted in a liquid phase in the presence or absence of the agent, the reaction products separated from unreacted components, and complexes detected; e.g. using an immobilized antibody specific for one of the binding components to anchor any complexes formed in solution, and a labeled antibody specific for the other partner to detect anchored complexes. Again, depending upon the order of addition of reactants to the liquid phase, test compounds that enable complex or that promote the stability of preformed complexes can be identified.

In an alternate embodiment, a homogeneous assay can be used. For example, a preformed complex of the A2M-based reagent and the interactive cellular or extracellular binding partner product is prepared in that either the target products or their binding partners are labeled, but the signal generated by the label is quenched due to complex formation. The addition of agent that favors the formation of the complex will result in the generation of a signal below the control value. In this way, agents that promote A2M-binding partner interaction can be identified.

In yet another aspect, the A2M-based reagent can be used as “bait proteins” in a two-hybrid assay or three-hybrid assay, to identify other proteins, which bind to or interact with A2M binding proteins and are involved in A2M activity. Such binding partners can be activators or inhibitors of signals or transcriptional control.

The two-hybrid system is based on the modular nature of most transcription factors, which consist of separable DNA-binding and activation domains. Briefly, the assay utilizes two different DNA constructs. In one construct, the gene that codes for a A2M binding partner is fused to a gene encoding the DNA binding domain of a known transcription factor (e.g. GAL-4). In the other construct, a DNA sequence, from a library of DNA sequences, that encodes an unidentified protein (“prey” or “sample”) is fused to a gene that codes for the activation domain of the known transcription factor. Alternatively the A2M can be the fused to the activator domain. If the “bait” and the “prey” proteins are able to interact, in vivo, forming a A2M dependent complex, the DNA-binding and activation domains of the transcription factor are brought into close proximity. This proximity allows transcription of a reporter gene (e.g. lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the A2M. In another embodiment, the two-hybrid assay is used to monitor an interaction between two components. The two hybrid assay can also be conducted in the presence of an agent to be screened, and the assay is used to determine whether the agent enhances or diminishes the interaction between the components.

In another embodiment, the assay for selecting compounds which interact with A2M can be a cell-based assay. Useful assays include assays in which a marker of matrix degradation is measured. The cell-based assay can include contacting a cell expressing a A2M-based reagent with a test compound and determining the ability of the test compound to modulate (e.g. stimulate or inhibit) an activity of A2M, and/or determine the ability of the agent to modulate expression of A2M, e.g. by detecting A2M-encoding nucleic acids (e.g. mRNA or cDNA) or related proteins in the cell. Determining the ability of the agent to modulate A2M activity can be accomplished, for example, by determining the ability of the A2M to bind to or interact with the test molecule, and by determining the ability of the test molecule to modulate matrix degradation. Cell-based systems can be used to identify compounds that decrease expression and/or activity and/or effect of a A2M. Such cells can be recombinant or non-recombinant, such as cell lines that express the A2M gene. In some embodiments, the cells can be recombinant or non-recombinant cells which express a A2M-binding partner. Exemplary systems include mammalian or yeast cells that express a A2M (for example from a recombinant nucleic acid). In utilizing such systems, cells are exposed to agents suspected of increasing expression and/or activity of a A2M. After exposure, the cells are assayed, for example, for A2M expression or activity. A cell can be from a stable cell line or a primary culture obtained from an organism (for example an organism treated with the agent).

In addition to cell-based and in vitro assay systems, non-human organisms, e.g. transgenic non-human organisms or a model organism, can also be used. A transgenic organism is one in which a heterologous DNA sequence is chromosomally integrated into the germ cells of the animal. A transgenic organism will also have the transgene integrated into the chromosomes of its somatic cells. Organisms of any species, including, but not limited to: yeast, worms, flies, fish, reptiles, birds, mammals (e.g. mice, rats, rabbits, guinea pigs, pigs, micro-pigs, and goats), and non-human primates (e.g. baboons, monkeys, chimpanzees) may be used in the methods described herein.

A transgenic cell or animal used in the methods of the invention can include a transgene that encodes A2M. The transgene can encode a protein that is normally exogenous to the transgenic cell or animal, including a human protein, e.g. a human A2M or one of its binding partner. The transgene can be linked to a heterologous or a native promoter. Methods of making transgenic cells and animals are known in the art.

In another assay format, the specific activity of A2M, normalized to a standard unit, may be assayed in a cell-free system, a cell line or a cell population that has been exposed to the agent to be tested and compared to an unexposed control cell-free system, cell line or cell population. The specific activity of an A2M-activating reagent can also be assessed using A2M-deficient systems (A2M knockout cells or animals).

Once the measurement has been made, it is extracted from the reaction vessel, and the value of the parameter of the A2M-based reagent is compared to a control value to determine the effect of the agent on A2M expression or activity. In an embodiment, the comparison can be made by an individual. In another embodiment, the comparison can be made in a comparison module. Such comparison module may comprise a processor and a memory card to perform an application. The processor may access the memory to retrieve data. The processor may be any device that can perform operations on data. Examples are a central processing unit (CPU), a front-end processor, a microprocessor, a graphics processing unit (PPU/VPU), a physics processing unit (PPU), a digital signal processor and a network processor. The application is coupled to the processor and configured to determine the effect of the agent on the parameter of the A2M-based reagent with respect to the control value. An output of this comparison may be transmitted to a display device. The memory, accessible by the processor, receives and stores data, such as measured parameters of the A2M-based reagent or any other information generated or used. The memory may be a main memory (such as a high speed Random Access Memory or RAM) or an auxiliary storage unit (such as a hard disk, a floppy disk or a magnetic tape drive). The memory may be any other type of memory (such as a Read-Only Memory or ROM) or optical storage media (such as a videodisc or a compact disc).

Once the comparison between the parameter of the A2M-based reagent and the control value is made, then it is possible to characterize the individual or the agent. This characterization is possible because, as shown herein, A2M is upregulated in individuals susceptible to and/or showing symptoms of osteonecrosis.

In an embodiment, the characterization can be made by an individual. In another embodiment, the characterization can be made with a processor and a memory card to perform an application. The processor may access the memory to retrieve data. The processor may be any device that can perform operations on data. Examples are a central processing unit (CPU), a front-end processor, a microprocessor, a graphics processing unit (PPU/VPU), a physics processing unit (PPU), a digital signal processor and a network processor. The application is coupled to the processor and configured to characterize the individual or the agent being screened. An output of this characterization may be transmitted to a display device. The memory, accessible by the processor, receives and stores data, such as measured parameters of the A2M-based reagent or any other information generated or used. The memory may be a main memory (such as a high speed Random Access Memory or RAM) or an auxiliary storage unit (such as a hard disk, a floppy disk or a magnetic tape drive). The memory may be any other type of memory (such as a Read-Only Memory or ROM) or optical storage media (such as a videodisc or a compact disc).

### Methods and Systems for the Characterization of a Usefulness of an Agent as a Therapy for Osteonecrosis

As shown herein, the level of expression of A2M is upregulated in the very first steps of osteonecrosis even when no symptoms are experienced by the afflicted individual. Consequently, because the level of expression of A2M is associated with the onset of osteonecrosis, the level of expression of A2M can be used to screen for potential agents for treating osteonecrosis. Agents that fail to increase A2M expression level or activity in the presence of corticosteroid will be considered useful as a therapeutic measure for osteonecrosis. In the methods and systems described for screening agents useful in the treatment of osteonecrosis, the A2M-based reagent is mixed first with a corticosteroid (to induce an osteogenesis-like state) and then with the agent. The level of the parameter of the A2M-based reagent is assessed prior to and after contact with the agent. These two levels are then compared to determine the usefulness of the agent to treatment of osteonecrosis. If the presence of the agent reverts the upregulation of the expression of the A2M-based reagent observed upon the addition of the corticosteroid, then it is determined that the agent is useful for the treatment (prevention to progression to later stages) of osteonecrosis.

In a first step, an agent to be screened is combined in a reaction vessel with an A2M-based reagent. The reaction vessel is designed as such that, upon the addition of a corticosteroid, the level of the parameter of the A2M-based reagent (in the absence of the agent) is upregulated. For screening applications, a suitable in vitro environment for the screening assay described herewith can be a cultured cell. Such cell should be able to maintain viability in culture. The cultured cell(s) should (i) express a polynucleotide encoding A2M or biologically active variant thereof (ii) express a A2M-encoding polynucleotide or variant thereof or related chimeric protein and/or (iii) comprise the A2M promoter region. If a primary cell culture is used, the cell may be isolated or in a tissue-like structure, for example, as part of a bone-like structure. A further suitable environment is a non-human model, such as an animal model. If the characterization of the agent occurs in a non-human model, then the model (such as a rodent) is administered with the agent. Various dosage and modes of administration maybe used to fully characterize the agent's ability to treat osteonecrosis.

Once the agent has been combined in the reaction vessel with the A2M-based reagent, a measurement or value of a parameter of the A2M-based reagent is made. This assessment may be made directly in the reaction vessel (by using a probe) or on a sample of such reaction vessel. The measurement of the parameter of the A2M-based reagent can be made either at the DNA level, the RNA level and/or the polypeptide level.

The measuring step can rely on the addition of a quantifier specific to the parameter to be assessed to the reaction vessel or a sample thereof. The quantifier can specifically bind to a parameter of a A2M-based reagent that is being assessed, such as, for example, a nucleotide product encoding A2M or a A2M polypeptide. In those instances, the amount of the quantifier that specifically bound (or that did not bind) to the A2M-based reagent can be determined to provide a measurement of the parameter of the A2M-based reagent. In another embodiment, the quantifier can be modified by a parameter of the A2M-based reagent. In this specific instance, the amount of modified (or unmodified) quantifier will be determined to provide a measurement of the parameter of the A2M-based reagent. In an embodiment, the signal of the quantifier can be provided by a label that is either directly or indirectly linked to a quantifier.

Various parameters of the A2M-based reagent can be measured. For example, when the A2M-based reagent is a A2M polypeptide or fragment thereof, the parameter that is measured can be the polypeptide biological activity (ability to bind to its cognate receptors and elicit cellular signaling, removal of OGP or inhibition of BMP-1), the polypeptide quantity and/or stability. When the A2M-based reagent is a nucleotide encoding a A2M polypeptide or fragment thereof, the parameter can be the level of expression or stability of the A2M-encoding nucleotide. Even though a single parameter is required to enable the characterization of the agent, it is also provided that more than one parameter of the A2M-based reagent may be measured.

If the measurement of the parameter is performed at the nucleotide level, then the transcription activity of the promoter associated with the A2M gene can be assessed. This assessment can be made, for example, by placing a reporter vector (such as a luciferase reporter based assay) in the presence of the A2M polypeptide (inside or outside a cell). Such reporter vectors can include, but are not limited to, the promoter region of the A2M gene (or fragment thereof) operably linked to a nucleotide encoding a reporter polypeptide (such as, for example, A2M, β-galactosidase, green-fluorescent protein, yellow-fluorescent protein, etc.). Upon the addition of the biological sample or the agent, the promotion of transcription from the promoter of the A2M gene is measured indirectly by measuring the transcription of the reporter polypeptide. In this particular embodiment, the quantifier is the reporter polypeptide and the signal associated to this quantifier that is being measured will vary upon the reporter polypeptide used. Alternatively or complementarily, the stability and/or the expression level of the A2M-encoding nucleotide can be assessed by quantifying the amount of a A2M-encoding nucleotide (for example using qPCR) or the stability of such nucleotide.

In one screening assay format, the expression of a nucleic acid encoding A2M in a cell or tissue sample is monitored directly by hybridization to the nucleic acids specific for A2M. In another assay format, cell lines or tissues can be exposed to the agent to be tested under appropriate conditions and time, and total RNA or mRNA isolated, optionally amplified, and quantified.

If the measurement of the parameter is performed at the polypeptide level, an assessment of A2M biological activity can be performed. A2M is a transcription factor that removes OGP, downregulates and/or inhibits BMP-1. As such, one of A2M's biological activity is to bind to other transcription regulators (also referred to as binding partners) as well as to bind to its target sequences.

Such evaluation can be made in vitro. The reaction mixture can include a binding partner or potentially interacting fragment thereof. This type of assay can be accomplished, for example, by coupling one of the components, with a label such that binding of the labeled component to the other can be determined by detecting the either directly or indirectly, and the radioisotope detected by direct counting of radioemission or by scintillation counting. Alternatively, a component can be enzymatically labeled with, for example, horseradish peroxidase, alkaline phosphatase, or luciferase, and the enzymatic label detected by determination of conversion of an appropriate substrate to product. Competition assays can also be used to evaluate a physical interaction between a test compound and a target.

In another assay format, A2M's activity can be indirectly measured by determining its ability to bind to its cognate receptor and elicit signaling from these receptors. A2M binds to at least two different cell surface receptors. The first A2M cell surface receptor is the low density lipoprotein receptor-related protein 1 (LRP1), involved in a broad range of proteins and is involved in many biological processes and diseases. The second A2M cell surface protein is glucose regulated protein of 78 kDa (GRP78) expressed in the ER, and plays an important role for the folding and maturation of proteins.

Cell-free screening assays usually involve preparing a reaction mixture of the target protein and the test compound under conditions and for a time sufficient to allow the two components to interact and bind, thus forming a complex that can be removed and/or detected.

The interaction between two molecules can also be detected, e.g. using a fluorescence assay in which at least one molecule is fluorescently labeled. One example of such an assay includes fluorescence energy transfer (FET or FRET for fluorescence resonance energy transfer). A fluorophore label on the first “donor” molecule is selected such that its emitted fluorescent energy will be absorbed by a fluorescent label on a second “acceptor” molecule, which in turn is able to fluoresce due to the absorbed energy. Alternately, the “donor” protein molecule may simply utilize the natural fluorescent energy of tryptophan residues. Labels are chosen that emit different wavelengths of light, such that the “acceptor” molecule label may be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. A FET binding event can be conveniently measured through standard fluorometric detection means well known in the art (e.g. using a fluorometer).

Another example of a fluorescence assay is fluorescence polarization (FP). For FP, only one component needs to be labeled. A binding interaction is detected by a change in molecular size of the labeled component. The size change alters the tumbling rate of the component in solution and is detected as a change in FP.

In another embodiment, the measuring step can rely on the use of real-time Biomolecular Interaction Analysis (BIA). “Surface plasmon resonance” or “BIA” detects biospecific interactions in real time, without labeling any of the interactants (e.g. BlAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the A2M-reagent is anchored onto a solid phase. The A2M-based reagent-related complexes anchored on the solid phase can be detected at the end of the reaction, e.g., the binding reaction. For example, the A2M-based reagent can be anchored onto a solid surface, and the test compound, (which is not anchored), can be labeled, either directly or indirectly, with detectable labels discussed herein. Examples of such solid phase include microtiter plates, test tubes, array slides, beads and micro-centrifuge tubes. In one embodiment, a A2M chimeric protein can be provided which adds a domain that allows one or both of the proteins to be bound to a matrix. Following incubation, the vessels are washed to remove any unbound components, the matrix immobilized in the case of beads, complex determined either directly or indirectly, for example, as described above. Alternatively, the complexes can be dissociated from the matrix, and the level of A2M binding or activity determined using standard techniques.

In order to conduct the assay, the non-immobilized component (agent or biological agent) is added to the coated surface containing the anchored component. After the reaction is complete, unreacted components are removed (e.g. by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface, e.g., using a labeled antibody specific for the immobilized component (the antibody, in turn, can be directly labeled or indirectly labeled with, e.g. a labeled anti-Ig antibody).

In one embodiment, this assay is performed utilizing antibodies specific to A2M or target molecules but which do not interfere with binding of the A2M to its target molecule. Such antibodies can be derivatized to the surface, and unbound target or the A2M-based reagent trapped on the surface by antibody conjugation. Methods for detecting such complexes, in addition to those described above for the GST-immobilized complexes, include immunodetection of complexes using antibodies reactive with the A2M-based reagent or target molecule, as well as enzyme-linked assays which rely on detecting an enzymatic activity associated with the A2M-based reagent or target molecule.

Alternatively, cell free assays can be conducted in a liquid phase. In such an assay, the reaction products are separated from unreacted components, by any of a number of standard techniques, including but not limited to: differential centrifugation; chromatography (gel filtration chromatography, ion-exchange chromatography) and/or electrophoresis. Such resins and chromatographic techniques are known to one skilled in the art. Further, fluorescence energy transfer may also be conveniently utilized, as described herein, to detect binding without further purification of the complex from solution.

To identify agents that facilitate with the interaction between the target product and its binding partner(s), for example, a reaction mixture containing the A2M-based reagent and the binding partner is prepared, under conditions and for a time sufficient, to allow the two products to form complex. In order to test if an agent which facilitates the interaction between A2M and its binding partner, the reaction mixture can be provided in the presence and absence of the test agent. The test agent can be initially included in the reaction mixture, or can be added at a time subsequent to the addition of the target and its cellular or extracellular binding partner. Control reaction mixtures are incubated without the test agent or with vehicle. The formation of any complexes between the target product and the cellular or extracellular binding partner is then detected. The formation of a complex in the reaction mixture containing the test compound, but not in the control reaction, indicates that the test agent facilitates the interaction of the A2M-based reagent and the interactive binding partner. In an embodiment, it is possible to detect the formation of the A2M-based complex indirectly by measuring the level of expression of a reporter gene whose expression is modulated by the presence (or absence) of the complex.

These assays can be conducted in a heterogeneous or homogeneous format. Heterogeneous assays involve anchoring either the A2M-based reagent or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the agents being tested. For example, test agents that interfere with the interaction between the A2M-based reagent and the binding partners, e.g. by competition, can be identified by conducting the reaction in the presence of the test substance. Alternatively, test agents that facilitates preformed complexes, can be tested by adding the test compound to the reaction mixture prior to complexes have been formed. The various formats are briefly described below.

In a heterogeneous assay system, either the A2M-based reagent or the binding partner, is anchored onto a solid surface (e.g. a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the agent. After the reaction is complete, unreacted components are removed (e.g. by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g. using a labeled antibody specific for the initially non-immobilized species (the antibody, in turn, can be directly labeled or indirectly labeled with, e.g. a labeled anti-Ig antibody). Depending upon the order of addition of reaction components, agents that enable complex formation or that promote the stability of preformed complexes can be detected.

Alternatively, the reaction can be conducted in a liquid phase in the presence or absence of the agent, the reaction products separated from unreacted components, and complexes detected; e.g. using an immobilized antibody specific for one of the binding components to anchor any complexes formed in solution, and a labeled antibody specific for the other partner to detect anchored complexes. Again, depending upon the order of addition of reactants to the liquid phase, test compounds that enable complex or that promote the stability of preformed complexes can be identified.

In an alternate embodiment, a homogeneous assay can be used. For example, a preformed complex of the A2M-based reagent and the interactive cellular or extracellular binding partner product is prepared in that either the target products or their binding partners are labeled, but the signal generated by the label is quenched due to complex formation. The addition of agent that favors the formation of the complex will result in the generation of a signal below the control value. In this way, agents that promote A2M-binding partner interaction can be identified.

In yet another aspect, the A2M-based reagent can be used as “bait proteins” in a two-hybrid assay or three-hybrid assay, to identify other proteins, which bind to or interact with A2M binding proteins and are involved in A2M activity. Such binding partners can be activators or inhibitors of signals or transcriptional control.

The two-hybrid system is based on the modular nature of most transcription factors, which consist of separable DNA-binding and activation domains. Briefly, the assay utilizes two different DNA constructs. In one construct, the gene that codes for a A2M binding partner is fused to a gene encoding the DNA binding domain of a known transcription factor (e.g. GAL-4). In the other construct, a DNA sequence, from a library of DNA sequences, that encodes an unidentified protein (“prey” or “sample”) is fused to a gene that codes for the activation domain of the known transcription factor. Alternatively the A2M can be the fused to the activator domain. If the “bait” and the “prey” proteins are able to interact, in vivo, forming a A2M dependent complex, the DNA-binding and activation domains of the transcription factor are brought into close proximity. This proximity allows transcription of a reporter gene (e.g. lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the A2M. In another embodiment, the two-hybrid assay is used to monitor an interaction between two components, the two hybrid assay can also be conducted in the presence of an agent to be screened, and the assay is used to determine whether the agent enhances or diminishes the interaction between the components.

In another embodiment, the assay for selecting compounds which interact with A2M can be a cell-based assay. Useful assays include assays in which a marker of matrix degradation is measured. The cell-based assay can include contacting a cell expressing a A2M-based reagent with a test compound and determining the ability of the test compound to modulate (e.g. stimulate or inhibit) an activity of a A2M, and/or determine the ability of the agent to modulate expression of a A2M, e.g. by detecting A2M-encoding nucleic acids (e.g. mRNA or cDNA) or related proteins in the cell. Determining the ability of the agent to modulate A2M activity can be accomplished, for example, by determining the ability of the A2M to bind to or interact with the test molecule, and by determining the ability of the test molecule to modulate matrix degradation. Cell-based systems can be used to identify compounds that decrease expression and/or activity and/or effect of a A2M. Such cells can be recombinant or non-recombinant, such as cell lines that express the A2M gene. In some embodiments, the cells can be recombinant or non-recombinant cells which express a A2M-binding partner. Exemplary systems include mammalian or yeast cells that express a A2M (for example from a recombinant nucleic acid). In utilizing such systems, cells are exposed to agents suspected of increasing expression and/or activity of a A2M. After exposure, the cells are assayed, for example, for A2M expression or activity. A cell can be from a stable cell line or a primary culture obtained from an organism (for example an organism treated with the agent).

In addition to cell-based and in vitro assay systems, non-human organisms, e.g. transgenic non-human organisms or a model organism, can also be used. A transgenic organism is one in which a heterologous DNA sequence is chromosomally integrated into the germ cells of the animal. A transgenic organism will also have the transgene integrated into the chromosomes of its somatic cells. Organisms of any species, including, but not limited to: yeast, worms, flies, fish, reptiles, birds, mammals (e.g. mice, rats, rabbits, guinea pigs, pigs, micro-pigs, and goats), and non-human primates (e.g. baboons, monkeys, chimpanzees) may be used in the methods described herein.

A transgenic cell or animal used in the methods of the invention can include a transgene that encodes A2M. The transgene can encode a protein that is normally exogenous to the transgenic cell or animal, including a human protein, e.g. a human A2M or one of its biding partner. The transgene can be linked to a heterologous or a native promoter. Methods of making transgenic cells and animals are known in the art.

In another assay format, the specific activity of A2M, normalized to a standard unit, may be assayed in a cell-free system, a cell line or a cell population that has been exposed to the agent to be tested and compared to an unexposed control cell-free system, cell line or cell population. The specific activity of an A2M-activating reagent can also be assessed using A2M-deficient systems (A2M knockout cells or animals).

Once the measurement has been made, it is extracted from the reaction vessel, and the value of the parameter of the A2M-based reagent is compared to a control value to determine the effect of the agent on A2M expression or activity. In an embodiment, the comparison can be made by an individual. In another embodiment, the comparison can be made in a comparison module. Such comparison module may comprise a processor and a memory card to perform an application. The processor may access the memory to retrieve data. The processor may be any device that can perform operations on data. Examples are a central processing unit (CPU), a front-end processor, a microprocessor, a graphics processing unit (PPU/VPU), a physics processing unit (PPU), a digital signal processor and a network processor. The application is coupled to the processor and configured to determine the effect of the agent on the parameter of the A2M-based reagent with respect to the control value. An output of this comparison may be transmitted to a display device. The memory, accessible by the processor, receives and stores data, such as measured parameters of the A2M-based reagent or any other information generated or used. The memory may be a main memory (such as a high speed Random Access Memory or RAM) or an auxiliary storage unit (such as a hard disk, a floppy disk or a magnetic tape drive). The memory may be any other type of memory (such as a Read-Only Memory or ROM) or optical storage media (such as a videodisc or a compact disc).

Once the comparison between the parameter of the A2M-based reagent and the control value is made, then it is possible to characterize the individual or the agent. This characterization is possible because, as shown herein, A2M is upregulated in individuals susceptible to and/or showing symptoms of osteonecrosis.

In an embodiment, the characterization can be made by an individual. In another embodiment, the characterization can be made with a processor and a memory card to perform an application. The processor may access the memory to retrieve data. The processor may be any device that can perform operations on data. Examples are a central processing unit (CPU), a front-end processor, a microprocessor, a graphics processing unit (PPU/VPU), a physics processing unit (PPU), a digital signal processor and a network processor. The application is coupled to the processor and configured to characterize the individual or the agent being screened. An output of this characterization may be transmitted to a display device. The memory, accessible by the processor, receives and stores data, such as measured parameters of the A2M-based reagent or any other information generated or used. The memory may be a main memory (such as a high speed Random Access Memory or RAM) or an auxiliary storage unit (such as a hard disk, a floppy disk or a magnetic tape drive). The memory may be any other type of memory (such as a Read-Only Memory or ROM) or optical storage media (such as a videodisc or a compact disc).

The present invention will be more readily understood by referring to the following examples which are given to illustrate the invention rather than to limit its scope.

### Example I

### Tissue A2M Expression in Steroid-Treated Animals

Experimental animals and their maintenance. Forty Wistar Kyoto (WKY) rats (ages 4 weeks old) were purchased from Charles River Laboratories (Pointe-Claire, QC, Canada). The rats were tagged and housed in plastic cages (2 to 4 animals per cage) under standard laboratory conditions with a 12-hour dark/12-hour light cycle, a constant temperature of 20° C., and humidity of 48%. Food and water were provided ad libitum with a standard rodent diet. The weight of the rats were followed before and after the implant of a prednisone pellet for the first 3 consecutive weeks and then every month until the end of the experiment. All experiments were conducted under an animal protocol approved by the McGill Animal Care Department.

Glucocorticoid Administration.

Slow-release prednisone pellets (Innovative Research of America, Sarasota Florida, USA) were implanted subcutaneously into 24 Wistar Kyoto rats (12 males and 12 females rats) at the age of 5 weeks. Each pellet was implanted underneath the skin on the lateral side of the neck by surgically making an incision and developing a pocket about 2 cm beyond the incision site. The pellet was placed in the pocket and the incision was sutured. Based on the manufacturer's instructions the pellet releases a constant dose of the drug subcutaneously. To maintain a constant dosage during the 6 month period of the experiment, second and third pellet implantations were performed using the same procedure at 2 and 3 months respectively. The average dose release from the pellet was equivalent to 1.5 mg/kg/day for the period of 6 months. The dose of corticosteroids and the duration of treatment were chosen based on clinical experience. For the control group, 16 age-matched Wistar Kyoto (8 males and 8 females) rats received placebo pellets (Innovative Research of America) introduced through the same surgical technique.

Histologic Examination.

The rats were sacrificed with an overdose of ketamine/xylazine at the age of 30 weeks. Tissue samples were obtained from the proximal femur containing the femoral head. Some samples were put in RNALater™ (QIAGEN Inc., Mississauga, ON, Canada) for RNA extraction and some samples were fixed for histological examination. Bone samples were fixed in 10% neutral buffered formalin, then decalcified in 4% ethylenediamine tetraacetic acid (pH 7.2) (Sigma-Aldrich, St. Louis, Mo., USA). The specimens were processed routinely and embedded in paraffin. Tissue samples were sectioned parasagitally with a rotary microtome at 4 to 5 microns thickness, stained with hematoxylin and eosin and evaluated by light microscopy.

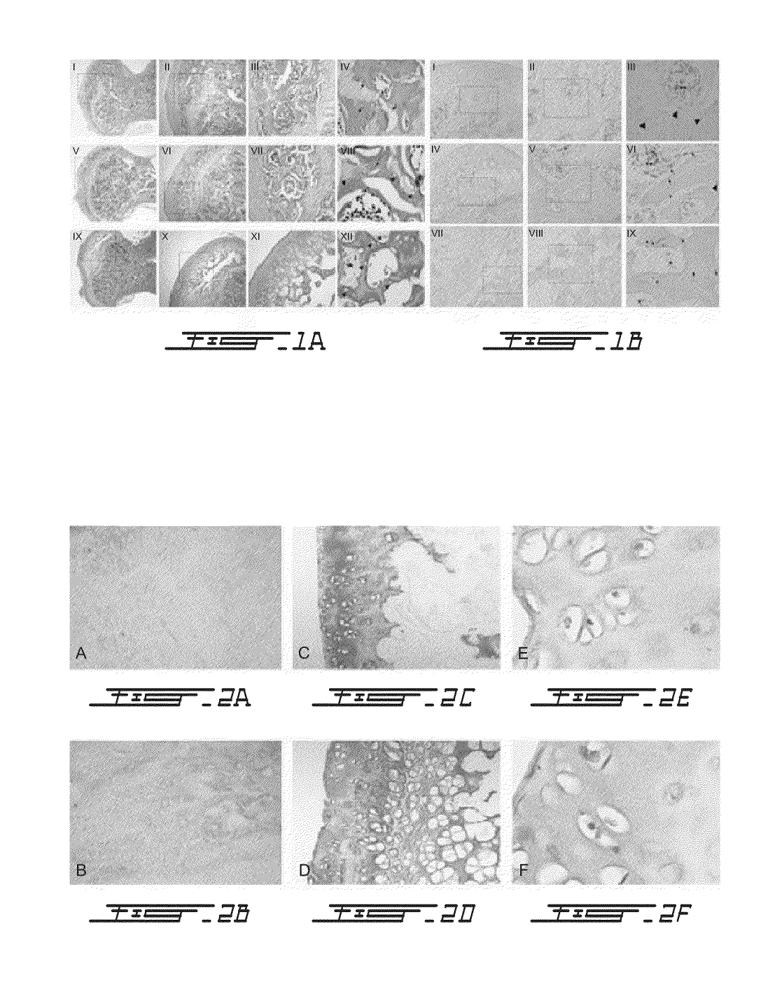

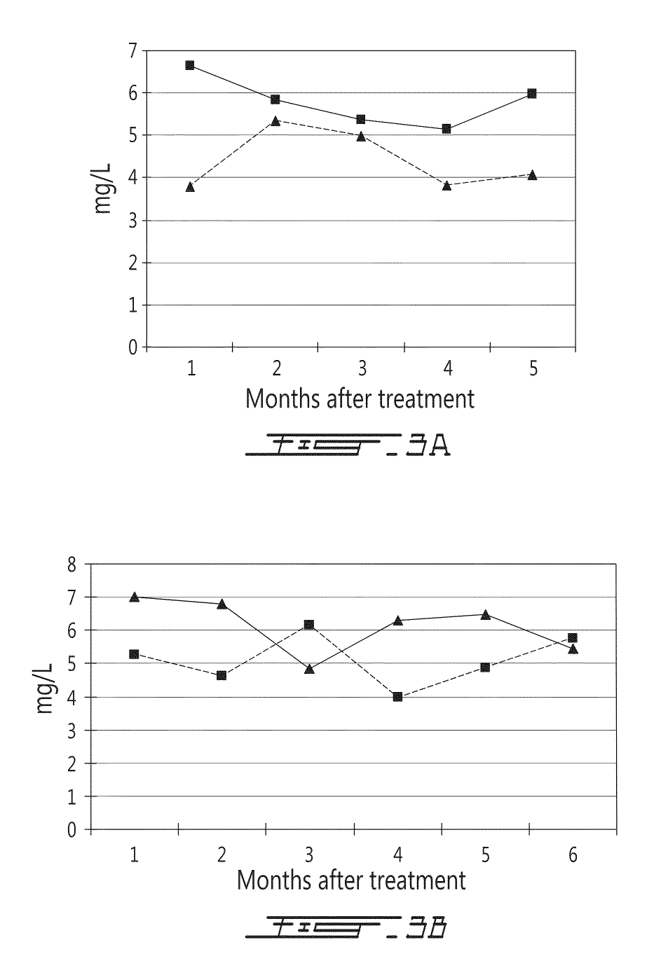

The tissue samples were analyzed in a blinded fashion by two experienced bone pathologists. The histological findings of an established ON are generally defined as dead trabeculae exhibiting empty lacunae with or without appositional bone formation [Wada et al., 2004], as shown in FIG. 1. While the development of ON proceeds through various clinically identifiable stages, it was preferable for this study to detect early as well as the late stages of the condition. With this objective in mind, the criteria of Arlet [Arlet, 1993] was adopted namely degeneration, necrosis, and disappearance of marrow cells as well as the nuclear disappearance and hypochromasia of trabecular osteocytes as early signs of ON [Arlet, 1993]. Early signs of ON was also considered when apoptosis occurred in the osteocytes and osteoblasts (FIG. 1). Positivity for apoptosis was defined by the authors as more than three osteocytes and/or osteoblasts recognized in a high magnification field based on previous studies [Weinstein et al, 2000; Shibahara et al., 2000]. The experiments were performed in triplicate. The histological findings of an established (late stage) ON were defined as empty lacunae. Early signs of ON was considered when apoptosis occurred in more than three osteocytes and/or osteoblasts recognized in a high magnification field (×200) (Table 1).

TABLE 1
Histological findings of avascular necrosis of the femoral head
(ON) in Wistar Kyoto rats.

| Sex | Treatment | No. of rats | OA/GC | OEL | EO | LO |
|---|---|---|---|---|---|---|
| Male | Placebo | 6 | 2 | 1 | 1 | 1 |
| Male | Prednisone | 7 | 5 | 1 | 4 | 1 |
| Female | Placebo | 5 | 1 | 0 | 1 | 0 |
| Female | Prednisone | 12 | 3 | 2 | 1 | 2 |

OA/GC: osteocyte apoptosis and/or ghost cell (a denucleated cell with an unstained center where the nucleus has been);
OEL: osteocyte empty lacunae;
EO: number of early stages of osteonecrosis;
LO: number of late stages of osteonecrosis

Measurement of Apoptosis in Undecalcified Bone Section.

Terminal dexoynucleotidyl transferase (TdT) mediated deoxyuridine triphosphate biotin nick end labeling (TUNEL) was used to detect fragmented DNA known to be associated with apoptotic cell death. TUNEL assay on paraffin-embedded tissue sections was performed with the DeadEnd™ Colorimetric TUNEL System (Promega, Madison, Wis., USA) as recommended by the manufacturer. Briefly, after deparaffinizing and permibilizing the tissue sections with proteinase K, the slides were incubated with the reaction mixture containing recombinant TdT and biotinylated nucleotide for 1 hour at 37° C. inside a humidified chamber. Labelled DNA was visualized with horseradish-peroxidase-labelled streptavidin using 3,3′-diaminobenzidine (DAB) as the chromogen. DNase I-treated tissue sections were used as positive controls. Negative controls for the study were sample slides processed using the same procedure but not treated with TdT Enzyme. All the slides were counterstained with 0.5% methyl green solution (0.5 g ethyl violet (Sigma-Aldrich) in 100 ml sodium acetate buffer, 0.1M and pH.4.2), cleared, mounted and evaluated by light microscopy.

RNA Extraction from Rat Bone Specimens.

Total RNA was extracted by combining the use of TRIzol® Reagent (Invitrogen, Carlsbad, Calif., USA) and RNeasy Mini kit (QIAGEN Inc.) followed by DNase I treatment (QIAGEN Inc.). Briefly, femoral head specimens were removed from RNALater™ and washed thoroughly with DEPCI-treated PBS. Femoral head specimens were placed in liquid nitrogen. The specimens were ground to a fine powder with a porcelain mortar and pestle. TRIzol® 1 ml was then added to each ground femoral head specimen. After vortexing for 1 min, the homogenized specimen was incubated for 5 min at room temperature (RT) and 0.2 ml chloroform (Sigma-Aldrich) was added per 1 ml of TRIzol®. After vortex use of 15 seconds the samples were incubated for 3 min at room temperature. The samples were then centrifuged at 12000×g for 15 min at 4° C. The aqueous phase was removed from each sample and one volume of ethanol was added to it and mixed thoroughly. Up to 700 μl of the sample including any precipitate that may have formed was transferred into an RNeasy™ Mini Spin Column. The column was then processed according to the RNeasy™ Mini kit manufacturer instruction. Any Genomic DNA contamination was removed by treating the samples with DNase I. The RNA quality was assessed using RNA 6000™ NanoChips with the Agilent 2100 Bioanalyzer™ (Agilent).

Affymetrix Exon Arrays.

Affymetrix GeneChip® Rat Exon 1.0 ST array interrogating over 850,000 exon clusters within the known and predicted transcribed regions of the entire genome and about one million probe sets was used. Affymetrix exon array was performed on 15 RNA samples of GC-treated and non-treated rats divided in 3 groups based on histological evaluation: Group 1—Placebo/ON (−); Group 2—GC-treated/ON (+) and Group 3—GC-treated/ON (−), each group consisting of 5 samples. Biotin labelled targets for the microarray experiment were prepared using 1 μg of total RNA. Ribosomal RNA was removed with the RiboMinus Human/Mouse Transcriptome™ Isolation Kit (Invitrogen, Eugene, Oreg., USA) and cDNA was synthesized using the GeneChip WT (Whole Transcript) Sense Target Labeling™ and Control Reagents kit as described by the manufacturer (Affymetrix, Santa Clara, Calif., USA). The sense cDNA was then fragmented by uracil DNA glycosylase and apurinic/apyrimidic endonuclease-1 and biotin-labeled with terminal deoxynucleotidyl transferase using the GeneChip WT Terminal labeling kit (Affymetrix). Hybridization was performed using 5 micrograms of biotinylated target, which was incubated with the GeneChip Rat Exon 1.0™ ST array (Affymetrix) at 45° C. for 16-20 h. After hybridization, non-specifically bound material was removed by washing and specifically bound target was detected using the GeneChip Hybridization, Wash and Stain kit, and the GeneChip Fluidics Station 450™ (Affymetrix). The arrays were scanned using the GeneChip Scanner 3000 7G™ (Affymetrix). An Affymetrix Power tools (Affymetrix) was used, R and in-house built Perl scripts to filter the background noise based on the “detection above background” results that is the detection metric generated by comparing Perfect Match probes to a distribution of background probes. Rat exon array data was analyzed and normalized based on the Iter-PLIER algorithm by using Affymetrix Power tools, R and in-house built Perl scripts. The genes with low signal (less than 100) were removed from the study. The differentially expressed genes were detected between three groups (G2 vs G1, G3 vs G2, and G3 vs G1) [p<0.05, Fold Change (FC)>1.5] using in house built R script, infer with t-test and adjusted with Benjamini and Hochberg FDR method [Benjamini et al, 1995].

Real-Time Polymerase Chain Reaction (SybrGreen RT-PCR).

Real-time PCR was carried out according to the protocol provided by the manufacturer for the QuantiTect SYBR®Green RT-PCR kit (QIAGEN Inc.). QuantiTect Primer™ Assays (Rn_A2 m_{—}1_SG, Rn_Col2a1_{—}1_SG, Rn_Mia1_{—}1_SG, Rn_Actb_1_SG) were provided by QIAGEN Inc. and a thermal cycler (Prism 7900™, Applied Biosystems, Foster City, Calif., USA) was used. The reaction was set up in 10 μl final volume applying the following conditions: cycling 50° C. (30 min), 95° C. (15 min) and for 45 cycles the conditions were 94° C. (15 Sec), 55° C. (30 Sec) and 72° C. (30 Sec). For the relative quantification of gene expression, the comparative threshold cycle (ACt) method was employed and normalized against β-Actin rRNA, which was measured by the same method. All PCR reactions were performed in triplicate. Control reactions were set up lacking reverse transcriptase to assess the level of contaminating genomic DNA.

Immunohistochemical (INC) Study.

Paraffin-embedded sections were placed at 60° C. for 15 min, incubated in xylene for 15 min, and then transferred sequentially into 100% ethanol, 95% ethanol, 70% ethanol, and 50% ethanol for 5 min at RT. Sections were rinsed in deionised water and the endogenous peroxidase activity was blocked with incubating sections in 3% H₂O₂ in distilled water for 5 min. The slides were washed in several changes of distilled water. Antigen was retrieved by incubating the slides in Digest-AIITM 3™ (Invitrogen Immunodetection, CA, USA) for 10 min. After several washes with PBS the slides were stained using R.T.U. Vectastain® Universal Quick kit (Vector Laboratories, Inc., CA, USA) according to the manufacturer's instructions. Several primary antibodies were used: 1:200 dilution of mouse anti-rat α-2-macroglobulin globulin monoclonal antibody (clone 129736, R&D Systems, Minneapolis, Minn., USA); prediluted mouse anti-rat collagen II antibody monoclonal antibody (Abcam Inc., Cambridge, Mass., USA) or 1:50 dilution of rabbit anti-rat melanoma inhibitory activity (MIA) polyclonal antibody (Santa Cruz, Biotechnology, Santa Cruz, Calif., USA). According to the manufacturer's instructions the secondary antibody is a prediluted biotinylated antibody manufactured in horse, which recognizes rabbit IgG, mouse IgG and goat IgG. The slides were counterstained with 0.5% methyl green solution as described before.

Statistical Analysis.

Data reported on microarray results utilized in-house Perl scripts with t-test and adjusted with B-H FDR method to examine differentially expressed genes between two groups [p<0.05, Fold Change (FC)>1.5]. RT-PCR results were given as the mean±SEM. Comparison between groups was made with Student's t-test. For small size samples Mann-Whitney U test was used since normal distribution of data was not assumed. Differences were considered significant at P values less than 0.05. Principle component analysis (PCA) was performed using R package to provide a global view of how the various sample groups were related.

Histological and Apoptosis Findings.

Histological findings displayed normal, early and late stages of ON based on the presence or absence of osteocytes in the lacunaes (FIG. 1A). The use of the TUNEL assay to detect apoptosis showed apoptotic osteocytes were located in the osteonecrotic samples without features of inflammation and visible necrosis, such as hyperemia, round cell infiltration, or lipid cyst formation. There was no appositional bone formation associated with granulation tissue around dead bone in keeping with the early stages of ON (FIG. 1B, IV-X). When the same TUNEL reaction was performed on control tissue (without prior digestion with DNase), a fewer number of cells (one or two) were labeled (FIG. 1B,

Microarray Analysis.

In the Affymetrix analysis, G2 replicates were compared with G1 and G3, separately, and G3 replicates were compared with G1 to generate a list of differentially expressed genes. The results were analyzed by a defined set of criteria in which the altered expression of a gene must have at least a change of ±1.5 fold (FC=fold change) and a p-value less than 0.05. These criteria resulted in the identification of 51 genes with significant modulation in G2 compared with G1 and 6 genes with significant modulation for G3 to G2 (Tables 4 and 5. They also identified 229 genes in G3 versus G1 (Table 2). In this table, only the genes with a change of ±1.8 fold (FC=fold change) are represented due to the exhaustive list of genes. Although rat exon array demonstrated a significant upregulation of 51 genes when comparing G2 to G1, alpha-2-macroglobulin gene was particularly found to be overexpressed when comparing steroid-treated Wistar Kyoto rats which had developed ON (G2) to placebo rats (G1) (FC=3.52, p=0.0005). Collagen type II alpha-1 (Col2A1) and Melanoma Inhibitory Activity-1 (MIA) genes were also found to be significantly overexpressed by exon array analysis (FC=2.52, p=0.0005 and FC=2.29, p=0.0008 respectively). The downregulation of some genes was not considered significant in terms of fold change compared to the upregulated genes. Significantly modulated genes were categorized into clusters according to their biological functions using DAVID, a functional annotation tool provided by National Institute of Allergy and Infectious Diseases-NIH. Modulated genes were grouped mainly into clusters of skeletal development, ossification and bone remodelling. Other functional classes significantly represented in the steroid-induced osteonecrosis included response to steroid stimulus response, apoptosis, blood vessel morphogenesis, vasculature development, cell growth, proliferation and differentiation associated genes. In comparison of G3 versus G1, A2M and Col2A1 were not significantly overexpressed whereas MIA was found to be the most up-regulated gene in that group comparison (FC=3.71, p=0.00).

TABLE 2
Differentially expressed genes from comparing group 3 (G3) versus 1 (G1).
Only genes with fold change above 1.8 have been shown.

| Annotation | | PV | FC |
|---|---|---|---|
| NM_030852 | melanoma inhibitory activity 1 | 0.0000 | +3.71 |
| NM_001002826 | murinoglobulin 2 | 0.0104 | +2.64 |
| NM_031808 | calpain 6 | 0.0002 | +2.62 |
| NM_019189 | hyaluronan and proteoglycan link protein 1 | 0.0000 | +2.61 |
| NM_001002826 | murinoglobulin 2 | 0.0007 | +2.37 |
| NM_001012034 | ADP-ribosyltransferase 3 | 0.0015 | +2.36 |
| NM_057104 | ectonucleotide | 0.0091 | +2.25 |
| | pyrophosphatase/phosphodiesterase 2 | | |
| NM_001009662 | carbonic anhydrase 8 | 0.0010 | +2.24 |
| NM_013191 | S100 protein, beta polypeptide, neural | 0.0002 | +2.24 |
| NM_138898 | phospholipase B | 0.0165 | +2.18 |
| NM_134432 | angiotensinogen (serpin peptidase inhibitor, | 0.0294 | +2.16 |
| | clade A, member 8) | | |
| NM_012620 | serine (or cysteine) peptidase inhibitor, clade | 0.0036 | +2.15 |
| | E, member 1 | | |
| NM_031828 | potassium large conductance calcium- | 0.0003 | +2.15 |
| | activated channel, subfamily M, alpha 1 | | |
| NM_133523 | matrix metallopeptidase 3 | 0.0162 | +2.15 |
| NM_138889 | cadherin 13 | 0.0011 | +2.14 |
| NM_133569 | angiopoietin-like 2 | 0.0000 | +2.14 |
| NM_001012163 | LIM and senescent cell antigen like domains 2 | 0.0048 | +2.13 |
| NM_001013213 | integrin beta 3 binding protein (beta3- | 0.0013 | +2.10 |
| | endonexin) | | |
| NM_198748 | scinderin | 0.0017 | +2.09 |
| NM_012497 | aldolase C | 0.0002 | +2.08 |
| NM_031694 | heat shock factor 2 | 0.0085 | +2.05 |
| NM_198768 | immunoglobulin superfamily, member 10 | 0.0015 | +2.03 |
| NM_053977 | cadherin 17 | 0.0062 | +2.02 |
| NM_001014060 | similar to SRY (sex determining region Y)-box | 0.0002 | +1.97 |
| | 5 isoform a | | |
| NM_012999 | proprotein convertase subtilisin/kexin type 6 | 0.0025 | +1.96 |
| NM_013080 | protein tyrosine phosphatase, receptor-type, Z | 0.0072 | +1.94 |
| | polypeptide 1 | | |
| NM_001002819 | glutamine-fructose-6-phosphate transaminase 2 | 0.0096 | +1.93 |
| BC079425 | hypothetical protein LOC654482 | 0.0009 | +1.92 |
| NM_031131 | transforming growth factor, beta 2 | 0.0013 | +1.91 |
| NM_022927 | midline 1 | 0.0070 | +1.90 |
| NM_181366 | G protein-coupled receptor 64 | 0.0010 | +1.90 |
| NM_022230 | stanniocalcin 2 | 0.0003 | +1.89 |
| NM_199398 | pannexin 3 | 0.0021 | +1.87 |
| NM_053605 | sphingomyelin phosphodiesterase 3, neutral | 0.0053 | +1.86 |
| NM_001009647 | mitochondrial ribosomal protein L16 | 0.0008 | +1.85 |
| NM_001077641 | phospholipase C, beta 1 | 0.0116 | +1.85 |
| NM_020073 | parathyroid hormone receptor 1 | 0.0016 | +1.83 |
| NM_017135 | adenylate kinase 3-like 1 | 0.0149 | +1.83 |
| NM_013000 | peptidylglycine alpha-amidating | 0.0065 | +1.82 |
| | monooxygenase | | |
| NM_001007656 | microtubule-associated protein, RP/EB family, | 0.0008 | +1.81 |
| | member 3 | | |
| NM_031590 | WNT1 inducible signaling pathway protein 2 | 0.0002 | +1.81 |
| NM_022382 | phosphodiesterase 4D interacting protein | 0.0127 | +1.80 |
| | (myomegalin) | | |
| NM_134327 | CD69 antigen | 0.0141 | −0.65 |
| NM_019295 | CD5 antigen | 0.0114 | −0.65 |
| NM_013121 | CD28 antigen | 0.0136 | −0.65 |
| NM_031147 | cold inducible RNA binding protein | 0.0025 | −0.64 |
| NM_001012226 | signal transducer and activator of transcription 4 | 0.0246 | −0.63 |
| NM_001008855 | RT1 class Ib gene, H2-TL-like, grc region (N3) | 0.0005 | −0.60 |
| NM_001012461 | deoxynucleotidyltransferase, terminal | 0.0140 | −0.59 |
| NM_173096 | myxovirus (influenza virus) resistance 1 | 0.0186 | −0.59 |
| NM_001009680 | 2′-5′ oligoadenylate synthetase 1I | 0.0039 | −0.58 |
| NM_001008836 | RT1 class I, CE13 | 0.0111 | −0.56 |
| NM_203410 | interferon, alpha-inducible protein 27-like | 0.0018 | −0.51 |

PV = p value,
FC = Fold change,
(+) = Positive Regulation,
(−) = Negative Regulation

Real Time PCR Verification of GeneChip Data.

From the microarray results, the 3 genes [α-2-macroglobulin (A2M), collagen type II alpha-1 (Col2A1), melanoma inhibitory activity-1 (MIA)] showing the highest upregulation or fold change were selected for validation by means of RT-PCR. The directional fold change was confirmed for all 3 genes and the correlation with microarray results was established. Some variations, however, were noted in the fold-change values demonstrated by real time PCR compared with values obtained by GeneChip analysis (for A2M, FC=3.52 with exon array and 5.85 with RT-PCR). Variations in fold change values between GeneChip and real time PCR might have been due to different methods of normalization and specificity/sensitivity of each method but the trends were the same for the 2 methods (differences with p values: 0.005-0.0009, Table 3).

TABLE 3
Correlation of gene expression comparing group 2 (G2) and 1 (G1) as
assessed by microarray and Real time PCR (p < 0.005 for all genes).

| | | Fold Change of a signal | |
|---|---|---|---|
| Annotation | | Microarray | Real time PCR |
| NM_012488 | alpha-2-macroglobulin | 3.52 | 5.85 |
| NM_012929 | collagen, type II, alpha 1 | 2.52 | 4.42 |
| NM_030852 | melanoma inhibitory | 2.29 | 2.80 |
| | activity 1 | | |

Immunohistochemistry.

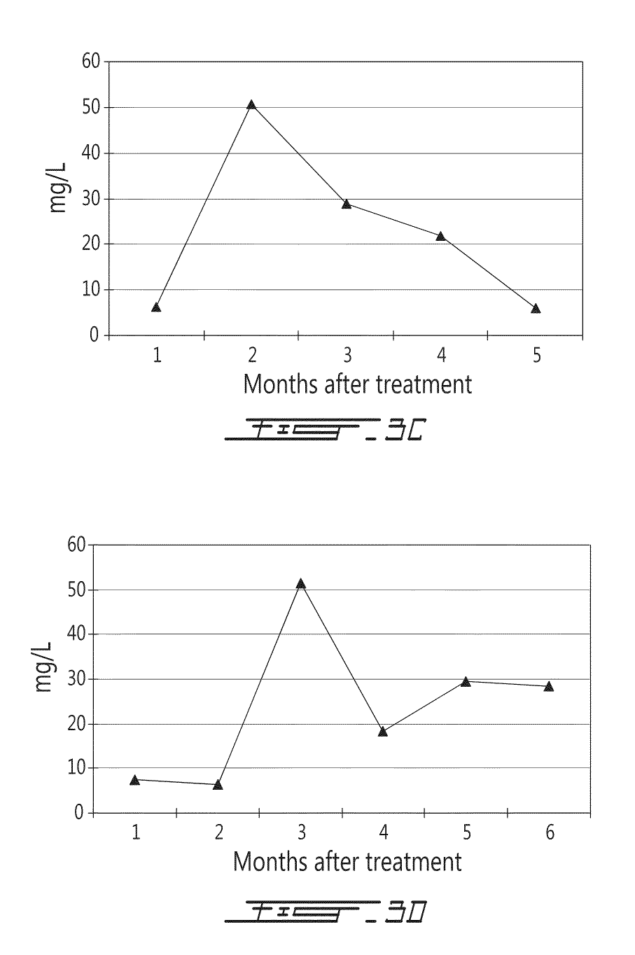

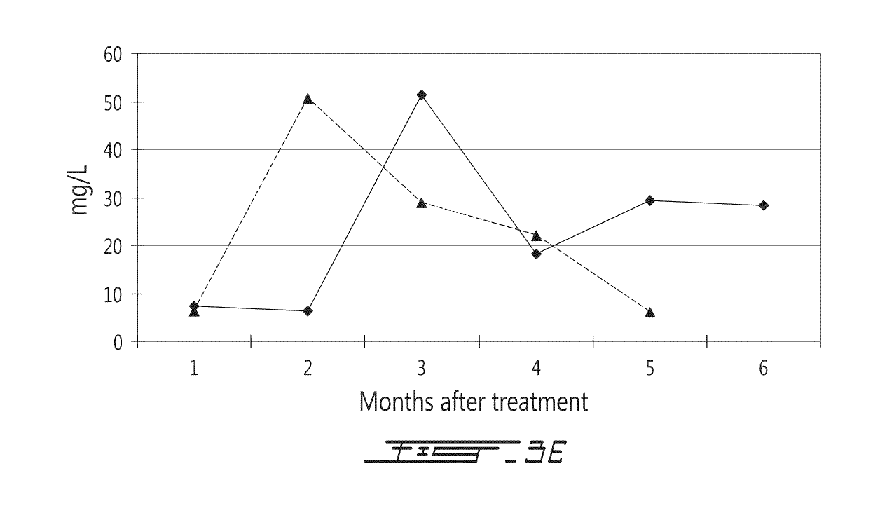

Immunohistochemistry staining was performed on the 3 candidate genes which showed the highest upregulation-A2M, Col2A1 and MIA-when comparing G2 to G1. Protein expression of A2M was shown to be increased in rats induced with steroids and developing ON (Group 2) as compared to the placebo rats without ON (Group 1) thus correlating with the mRNA expression levels from GeneChip analysis and RT-PCR method (FIG. 2). Notably, immunohistochemical findings for the 2 other genes of interest (COL2A1 and MIA) failed to show enhanced protein expression with this method.

The early events in the pathogenesis of ON are incompletely understood due to a typically late diagnosis after fracture and collapse of the femoral head. Besides bone marrow changes, evidence has shown that apoptosis is involved in the early stages of steroid-induced osteonecrosis. Weinstein et al. reported that the number of apoptotic bone cells increased significantly in mice after steroid administration [Weinstein et al., 1998]. Recent studies have shown apoptotic cells in clinical and animal models of GC-induced ANFH [Shibahara et al., 2000; Kabata et al., 2000; Weinstein et al., 2000].

In previous studies, an inbred rat (WKY) was characterized as susceptible to develop steroid-induced osteonecrosis [Kerachian et al., 2007]. It is possible that this strain of rats has genetically predisposing factors to develop ON and additional risk exposures (GC) will facilitate the development of the disease. In this animal model, prednisone administration enhanced the incidence of the disease in up to 75% (6/8) of the male WKY rats, suggesting it is a suitable model. In addition, WKY rats started to receive continuous steroid dosage released from the pellets at the age of 5 weeks for a duration of 25 weeks. Harvest at 6 months showed classical histological signs of early ON.

For the Affymetrix GeneChip findings, comparison of G2 versus G1 indicated that multiple pathological reactions occurred. According to the functional annotation tool (DAVID), modulated genes in the comparison of G2 and G1 (Table 4) were grouped mainly into skeletal development, ossification and bone remodelling. Functional clusters of genes were significantly represented by steroid stimulus response, apoptosis, blood vessel morphogenesis, vasculature development, coagulation-related, cell growth, proliferation and differentiation associated genes.

TABLE 4
Differentially expressed genes from comparing Group 2 (G2) versus Group 1
(G1).

| Annotation | | PV | FC |
|---|---|---|---|
| NM_012488 | alpha-2-macroglobulin | 0.0005 | +3.52 |
| NM_012929 | collagen, type II, alpha 1 | 0.0005 | +2.52 |
| NM_030852 | melanoma inhibitory activity 1 | 0.0008 | +2.29 |
| NM_033499 | scrapie responsive gene 1 | 0.0054 | +2.08 |
| NM_017094 | Growth hormone receptor | 0.0142 | +1.93 |
| NM_053669 | SH2B adaptor protein 2 | 0.0213 | +1.89 |
| NM_080698 | fibromodulin | 0.0099 | +1.87 |
| NM_133523 | Matrix metallopeptidase 3 | 0.0034 | +1.87 |
| NM_012999 | Proprotein convertase subtilisin/kexin type 6 | 0.0117 | +1.80 |
| NM_138889 | cadherin 13 | 0.0049 | +1.77 |
| NM_031808 | calpain 6 | 0.0086 | +1.73 |
| NM_001002826 | murinoglobulin 2 | 0.0022 | +1.72 |
| NM_145776 | Solute carrier family 38, member 3 | 0.0040 | +1.71 |
| NM_012846 | fibroblast growth factor 1 | 0.0441 | +1.70 |
| NM_017058 | vitamin D receptor | 0.0065 | +1.69 |
| NM_001009662 | carbonic anhydrase 8 | 0.0275 | +1.68 |
| NM_031590 | WNT1 inducible signaling pathway | 0.0105 | +1.67 |
| | protein 2 | | |
| NM_012587 | integrin binding sialoprotein | 0.0276 | +1.66 |
| NM_053816 | calcitonin receptor | 0.0316 | +1.63 |
| NM_013191 | S100 protein, beta polypeptide, neural | 0.0123 | +1.62 |
| NM_031828 | potassium large conductance calcium- | 0.0018 | +1.62 |
| | activated channel, subfamily M, alpha | | |
| | member 1 | | |
| NM_133569 | angiopoietin-like 2 | 0 | +1.62 |
| NM_199398 | pannexin 3 | 0.0032 | +1.62 |
| NM_053605 | sphingomyelin phosphodiesterase 3, | 0.0126 | +1.62 |
| | neutral | | |
| NM_170668 | Solute carrier family 13 (sodium- | 0.0167 | +1.60 |
| | dependent citrate transporter), member | | |
| NM_053977 | cadherin 17 | 0.0233 | +1.60 |
| NM_199407 | unc-5 homolog C (C. elegans) | 0.0002 | +1.60 |
| NM_012620 | Serine (or cysteine) peptidase inhibitor, | 0.0003 | +1.60 |
| | clade E, member 1 | | |
| | (also designated plasminogen activator | | |
| | inhibitor-1 or PAI-1) | | |
| NM_022667 | Solute carrier organic anion transporter | 0.0055 | +1.59 |
| | family, member 2a1 | | |
| NM_001034009 | melanoma cell adhesion molecule | 0.0032 | +1.58 |
| NM_053288 | orosomucoid 1 | 0.0236 | +1.57 |
| NM_031131 | transforming growth factor, beta 2 | 0.0015 | +1.57 |
| NM_013059 | alkaline phosphatase, liver/bone/kidney | 0.0218 | +1.57 |
| NM_133303 | basic helix-loop-helix domain | 0.0114 | +1.56 |
| | containing, class B3 | | |
| NM_198768 | immunoglobulin superfamily, member | 0.0467 | +1.55 |
| | 10 | | |
| NM_001017479 | transmembrane protein 100 | 0.0431 | +1.54 |
| NM_020073 | parathyroid hormone receptor 1 | 0.0370 | +1.54 |
| NM_024400 | a disintegrin-like and metallopeptidase | 0.0086 | +1.54 |
| | (reprolysin type) with thrombospondin | | |
| | type 1 motif, 1 | | |
| NM_001014043 | sphingomyelin synthase 2 | 0.0131 | +1.53 |
| NM_023970 | transient receptor potential cation | 0.0219 | +1.52 |
| | channel, subfamily V, member 4 | | |
| NM_020656 | Parvin, alpha | 0.0072 | +1.52 |
| NM_175578 | regulator of calcineurin 2 | 0.0390 | +1.52 |
| NM_031655 | Latexin | 0.0080 | +1.52 |
| NM_001013218 | receptor accessory protein 6 | 0.0045 | +1.52 |
| NM_001005562 | cAMP responsive element binding | 0.0376 | +1.50 |
| | protein 3-like 1 | | |
| NM_001017496 | chemokine (C—X—C motif) ligand 13 | 0.0140 | −0.55 |
| ENSRNOT00000060250 | similar to T-cell receptor alpha chain | 0.0154 | −0.64 |
| | precursor V and C regions (TRA29) | | |
| NM_203410 | interferon, alpha-inducible protein 27- | 0.0325 | −0.64 |
| | like | | |
| NM_001008836 | RT1-CE13 // RT1 class I, CE13 | 0.0157 | −0.64 |
| NM_001002280 | MAS-related GPR, member X2 | 0.0021 | −0.66 |
| NM_001008855 | RT1 class Ib gene, H2-TL-like, grc | 0.0350 | −0.67 |
| | region (N3) | | |

V = p value,
FC = Fold change,
(+) = Positive Regulation,
(−) = Negative Regulation

The expression of steroid stimulus response genes (A2M, alkaline phosphatase, tissue-nonspecific, transforming growth factor beta 2 and potassium large conductance calcium-activated channel, subfamily m, alpha member 1) were, as predicted, altered significantly. Previous in vivo and in vitro models as well as clinical studies showed that steroids induce apoptosis in osteoblasts and osteocytes. Amongst the 51 differentially regulated genes identified in our gene array analysis (Table 4), five genes [S100 protein-beta polypeptide, transforming growth factor-beta 2, vitamin D receptor, unc-5 homolog c (C. elegans) and growth hormone receptor] are in fact components of the apoptosis pathway.

The process of apoptosis can be directly induced by steroids but is also related to thrombosis in the blood vessels of the femoral head. In fact, the vascular hypothesis (regional endothelial bed dysfunction) appears to be relevant in the pathogenesis of ON. Damage or activation of femoral head endothelial cells results in abnormal blood coagulation and thrombi formation. Due to heterogeneity of the phenotype expression between endothelial cells in the body, a local endothelial cell dysfunction can occur where the femoral head endothelial cells react differently to the ON risk factors (GCs) than other endothelial cells in the body. In keeping with the theory of endothelial cell activation having a role in ON, coagulation-related gene expression in particular serine (or cysteine) peptidase inhibitor, Glade E, member 1 also named plasminogen activator inhibitor 1 (PAI-1), a serine protease inhibitor that is synthesized and released by endothelial cells in the blood, was shown to be significantly over-expressed in this study. An increase in PAI-1 suppresses the generation of plasmin resulting in hypofibrinolysis and a relative hypercoagulable state. Decreased fibrinolytic activity, which may be a consequence of increased PAI-1, has been described in patients with ON, although a few studies have reported that there were no significant differences in the levels of thrombotic and fibrinolytic factors.

Similarly, the findings presented herein indicate that several genes involved in the dynamic remodelling structure of the femoral head are also shown to be differentially expressed in ON (Table 4). Clinically this may be relevant in that if the balance between degradation and repair (bone remodelling) becomes shifted to degradation and bone loss by the effect of GC, a failure of structural integrity at the subchondral region of bone with collapse could occur.

In the present study, results showed A2M gene expression to be the most significantly upregulated gene when comparing G2 to G1. Correlation was obtained at the microarray, RT-PCR as well as the protein level as demonstrated by IHC study results. Most importantly, A2M was not significantly upregulated when comparing G3 to G1. A2M is a plasma-derived matrix metalloproteinase inhibitor which obstructs cartilage degradation induced by matrix metalloproteinases.

Without wishing to be bound to theory, the literature supports the role of corticosteroids in the modulation of A2M expression. In both reports, corticosteroids were shown to enhance A2M levels. A2M is reported as being implicated in cartilage degradation, and as an osteogenic growth peptide (OGP)—binding protein. Activated A2M may thus participate in the removal of OGP from the system. Additional reports suggest inhibition of BMP-1 (bone morphogenic protein-1) by A2M. A2M has also been identified on the luminal surface of endothelial cells in sections of normal human arteries and veins. A2M has also been implicated in hemostasis as a regulator of thrombin and in the development of thromboembolism in children. Together, all these findings suggest that A2M shares haemostatic, cartilaginous and osteogenic properties and may have a potential role in the development of early steroid-induced ON. Determination of whether A2M over-expression in our study is either the result or the cause of the apoptosis found in our rats developing early ON following administration of steroids, will require further study.

Two other genes of interest, Col2A1 and MIA, were also shown to be over-expressed significantly by microarray analysis and RT-PCR results but immunohistochemical study failed to show an increased cell surface expression of these genes.

Comparing the gene profiling of G3 versus G2, 6 genes stood out in our analyses (Table 5). Although G3 animals have not developed ON, their gene profile reflects inhibition of osteoblast proliferation, differentiation and osteoclast activation. Perhaps most osteogenic cells in this group have not gone through the apoptotic phase and there are more viable cells expressing these molecules in comparison to G2. Differences could also be explained in that gene expression analysis findings are supportive of a result effect indicating steroid treatment and a disease effect affecting the apoptotic process, are involved in the early stages of ON. Secondly, a genetic variation based on differences in transcription and translation could provide an explanation for the phenotypic differences found in our study. Thirdly, epigenetic variation, resulting from the interaction between the genotype and the environment, is also a potential process that could explain the findings that not all treated animals developed early ON when submitted to the same experimental conditions. Also, any of the genes listed in the comparison of G3 to G2 (Table 5) with the exception of MIA, could have a protective effect against the development of steroid-induced early ON. Similarly, the absence of A2M over-expression in that same group comparison G3 to G2, and in group comparison G3 to G1 is consistent with the phenotypic absence of early ON in rats representing G3.

TABLE 5
Differentially expressed genes from comparing group 3
(G3) versus 2 (G2).

| Annotation | | PV | FC |
|---|---|---|---|
| NM_001012357 | chemokine (C-C motif) ligand 9 | 0.0371 | +1.86 |
| NM_013153 | hyaluronan synthase 2 | 0.0103 | +1.70 |
| NM_030852 | melanoma inhibitory activity 1 | 0.0082 | +1.62 |
| NM_001012072 | protein phosphatase 1, | 0.0411 | +1.58 |
| | regulatory (inhibitor) subunit 3C | | |
| NM_001009639 | tubulin polymerization-promoting | 0.0243 | +1.56 |
| | protein family member 3 | | |
| NM_012497 | aldolase C | 0.0155 | +1.54 |

PV = p value,
FC = Fold change,
(+) = Positive Regulation,
(−) = Negative Regulation

In summary, it is suggested that multiple pathological reactions occur during ON. Genetic predisposition contributes to the development of ON. There is normally a balance between degenerative and regenerative molecules in the bone environment of the femoral head. GCs may trigger a degenerative process as well as inhibit the repair. In this study, several molecules are significantly upregulated and could be involved in the pathogenesis of ON. However, only A2M gene over-expression has been consistently found at the microarray, RT-PCR and protein level for the 3 genes showing the most significant upregulation. Besides, A2M was not significantly upregulated in rats administered steroids but without developing the disease. Thus, A2M seems to be a possible biomarker more of ON itself (induced by steroids) than a marker of steroids alone. It remains to be determined in which specific pathway (although likely in the endothelial cell activation and/or the apoptosis pathway) and at which level, the effect of this gene occurs in corticosteroid-induced ON. Identifying its role within a specific pathway will likely lead to a better understanding of the molecular events that follow the administration of corticosteroids and subsequent irreversible necrosis and bone collapse. Obviously, investigation of the use of A2M as a potential marker for the early warning of ON should be carried out.

### Example II

### Blood Expression of A2M in Steroid-Treated Animals

Experimental Animals and their Maintenance.

Eighteen Wistar Kyoto (WKY) rats (ages 12-16 weeks old) and eighteen Fisher (ages 12-16 weeks old) were purchased from Charles River Laboratories (Pointe-Claire, QC, Canada). The rats were tagged and housed in plastic cages (2 to 4 animals per cage) under standard laboratory conditions with a 12-hour dark/12-hour light cycle, a constant temperature of 20° C., and humidity of 48%. Food and water were provided ad libitum with a standard rodent diet. The weight of the rats were followed before and after the implant of a prednisone pellet for the first 3 consecutive weeks and then every month until the end of the experiment. All experiments were conducted under an animal protocol approved by the McGill Animal Care Department.

Glucocorticoid Administration.

Slow-release prednisone pellets (Innovative Research of America, Sarasota Florida, USA) were implanted subcutaneously into 12 Wistar Kyoto rats (all males) and 12 Fisher rats (all males) at the age of 8-12 weeks. Each pellet was implanted underneath the skin on the lateral side of the neck by surgically making an incision and developing a pocket about 2 cm beyond the incision site. The pellet was placed in the pocket and the incision was sutured. Based on the manufacturer's instructions the pellet releases a constant dose of the drug subcutaneously. To maintain a constant dosage during the 6 month period of the experiment, second and third pellet implantations were performed using the same procedure at 2 and 3 months respectively. The average dose release from the pellet was equivalent to 1.5 mg/kg/day for the period of 6 months. The dose of corticosteroids and the duration of treatment were chosen based on clinical experience. For the control group, 6 age-matched Wistar Kyoto (all males) and 6 age-matched Fisher (all males) rats received placebo pellets (Innovative Research of America) introduced through the same surgical technique.

Blood Analysis.

All rats were weighed upon their entry into the animal facility and were then weighed on a weekly basis until the end of the experimental period. Serum glucose measurements were performed at 2 week intervals via saphenous venipuncture using a portable glucometer (Contour™ glucometer, Bayer). Furthermore, 1 ml of whole blood was collected from each animal once a month via sublingual venipuncture under general anesthesia following a previously described technique. Following venipuncture, the blood was transferred to standard EDTA tubes, centrifuged for 8 minutes at 3600 rpm and the serum was separated and initially stored at −20° Celsius. Monthly serum A2M concentrations were determined using a Rat Alpha 2-Marcoglobulin Enzyme Linked Immunoassay (ELISA) (Kamiya Biomedical Company, Seattle, USA) according to the manufacturer's instructions. This measurement was performed by an author blinded to treatment allocation.

Histologic Examination.

The rats were sacrificed with an overdose of ketamine/xylazine at the age of 32-36 weeks. Tissue samples were obtained from the proximal femur containing the femoral head. Some samples were put in RNALater™ (QIAGEN Inc., Mississauga, ON, Canada) for RNA extraction and some samples were fixed for histological examination. Bone samples were fixed in 10% neutral buffered formalin, then decalcified in 4% ethylenediamine tetraacetic acid (pH 7.2) (Sigma-Aldrich, St. Louis, Mo., USA). The specimens were processed routinely and embedded in paraffin. Tissue samples were sectioned parasagitally with a rotary microtome at 4 to 5 microns thickness, stained with hematoxylin and eosin and evaluated by light microscopy.

The tissue samples were analyzed in a blinded fashion by two experienced bone pathologists. The histological findings of an established ON are generally defined as dead trabeculae exhibiting empty lacunae with or without appositional bone formation [Wada et al., 2004]. While the development of ON proceeds through various clinically identifiable stages, it was preferable for this study to detect early as well as the late stages of the condition. With this objective in mind, the criteria of Arlet [Arlet, 1993] was adopted namely degeneration, necrosis, and disappearance of marrow cells as well as the nuclear disappearance and hypochromasia of trabecular osteocytes as early signs of ON [Arlet, 1993]. Early signs of ON was also considered when apoptosis occurred in the osteocytes and osteoblasts. Positivity for apoptosis was defined by the authors as more than three osteocytes and/or osteoblasts recognized in a high magnification field based on previous studies [Weinstein et al, 2000; Shibahara et al., 2000].

Measurement of Apoptosis in Undecalcified Bone Section.

Terminal dexoynucleotidyl transferase (TdT) mediated deoxyuridine triphosphate biotin nick end labeling (TUNEL) was used to detect fragmented DNA known to be associated with apoptotic cell death. TUNEL assay on paraffin-embedded tissue sections was performed with the DeadEnd™ Colorimetric TUNEL System (Promega, Madison, Wis., USA) as recommended by the manufacturer. Briefly, after deparaffinizing and permibilizing the tissue sections with proteinase K, the slides were incubated with the reaction mixture containing recombinant TdT and biotinylated nucleotide for 1 hour at 37° C. inside a humidified chamber. Labelled DNA was visualized with horseradish-peroxidase-labelled streptavidin using 3,3′-diaminobenzidine (DAB) as the chromogen. DNase I-treated tissue sections were used as positive controls. Negative controls for the study were sample slides processed using the same procedure but not treated with TdT Enzyme. All the slides were counterstained with 0.5% methyl green solution (0.5 g ethyl violet (Sigma-Aldrich) in 100 ml sodium acetate buffer, 0.1M and pH.4.2), cleared, mounted and evaluated by light microscopy.

Cell Culture and Treatment.

HUVECs were purchased from Lonza (Walkersville, Md., USA) and cultured in endothelial basal medium (EBM-2media (Lonza) supplemented with microvascular additives (excluding hydrocortisone): human endothelial growth factor, 5% fetal bovine serum (FBS), vascular endothelial growth factor, human fibroblast growth factor type B, R3 insulin-like growth factor 1, ascorbic acid, heparin and the appropriate antibiotics gentamicin and amphotericin B (as per the supplier's protocol). HUVECs cells were cultured to near confluence (80%), trypsinized and seeded on 6 wells plates (105 cells/well). After 24 h incubation, cells were treated with dexamethasone (0.25 to 1 mM, Sigma-Aldrich, St-Louis, Mo., USA) for different time periods (between 4h to 48h). For experiments relating to the signaling pathways, different inhibitors were used: PD98059 (20 μM, Sigma) a MAPK inhibitor; withaferin (1 μM, Tocris, Bristol, UK), an NFkB PI3K inhibitor, and thrombin (1.5 U/ml; Calbiochem, San Diego, Calif., USA) an NFkB agonist. After incubation cells were harvested and RNA extracted using Trisol® (Invitrogen, Burlington, ON, Canada). RNA were then reverse transcribed using high capacity RNA to cDNA kit (Applied biosystems, Carlsbad, Calif., USA). A2M expression levels were then determined by qPCR using Taqman probes and master mix (Applied Biosystems)

Femur Harvesting and Micro-Computerized Tomography Scanning.

At the conclusion of the 180 day experimental period, all prednisone and placebo animals were euthanized via brief isoflurane exposure followed by cardiac puncture. Immediately following euthanasia, both femurs in each animal were dissected, with care taken to preserve the integrity of the femoral cartilage during hip disarticulation. The femurs were then osteotomized at the mid-diaphysis and the proximal portion was immersed initially in 4% paraformaldehyde for 24 hours, washed with sterile phosphate buffered saline (PBS) for the following 48 hours, and then preserved in PBS at −5° Celsius.

Micro computed-tomography (μCT) scanning was performed on each harvested femur using a Skyscan1172 instrument equipped with a 1.3 Mp camera (Skyscan, Kontich, Belgium). Using an energy source of 80 kV and 100 μA, images were captured at a rotation step of 0.45° between frames using a 0.5 mm aluminum filter at a magnification of 5 μm per pixel. 2-dimensional serial cross sections were assembled into 3D reconstructions and analyzed using Skyscan™ software (CTAn version 2.0.0.1) supplied with the instrument. Cross sections were oriented such that the viewing plane was perpendicular to the central portion of the proximal femoral growth plate.

In the present example, an analysis of the epiphyseal trabecular bone adjacent to the subchondral plate was performed. Upon identifying the radiolucent borders of the growth plate, a rectangular cubic volume of interest, measuring 1 mm×1 mm and a depth of 0.5 mm was drawn. This shape permitted the largest area of epiphyseal bone to be measured without artefact from the adjacent rodent growth plate or cortical cap. A threshold of 40% maximum grayscale (80/255) was used to segment bone from nonbone.

Four widely utilized morphometric indices measuring bone volume and bone connectivity were measured from the binarized 3-dimensional volume of interest. The fractional trabecular bone volume (BVF), which represents the percentage of trabecular bone volume relative to the total tissue volume, was measured. Total bone porosity (TBP) was also measured. Bone connectivity was assessed by measuring the number of bony trabecular present (Trabecular number; Tb.N), and the absolute value of the trabecular pattern factor, an index of connectivity that is based on the number of convex and concave surfaces within bone.

Statistical Analysis.

Analysis of variance for repeated measures was used to examine both change in body weight and serum glucose, with treatment with placebo or glucocorticoids tested as independent factors. If Mauchly's test of sphericity was violated at the 0.05 level, the degrees of freedom were adjusted using the Huynh-Feldt correct to calculate the critical t-statistic. Comparison between groups for micro CT bone morphometric indices were performed using a two-tailed Student's t-test. Differences were considered significant at P-values less than 0.05. [Insert A2M statistics here and regression analysis for A2M→histology here]. All statistical tests were performed using SPSS Statistical Software version 19.0 (SPSS Inc., Chicago, USA).

Table 6 provides a summary of histopathology study and TUNEL assay on bilateral femora of Fisher and Wistar Kyoto (WK) rats which received control (placebo) and steroid treatment for 5 and 6 months, respectively. After rats were euthanized, bilateral femora were isolated and processed for paraffin sectioning. Histopathology was conducted on H & E stained sections by two pathologists and avascular necrosis (AVN) was judged to be present (+) when necrosis of the medullary haematopoietic cells or fat cells, or empty lacunae or condensed nuclei in osteocytes were noted. TUNEL assay was performed on sections from samples which are AVN (+), or from any one of the hips from rats that were bilateral AVN (−), with in Situ Cell Death Detection Kit™ (Roche). TUNEL stained sections were examined by light microscopy with 20X lense, in area near the growth plate of femoral head, and if less than 5 TUNEL positive cells were observed in only one view (field), the sample was noted as TUNEL(+); if more than 5 positive cells were observed in one or more than two views (fields), sample was noted as TUNEL(++). Based on these observations, apoptotic cells (TUNEL positive cells) could be found in the bilateral femora of both Fisher and Wistar Kyoto placebo-treated rats, and apoptosis was more profound in rats receiving steroid treatment. The results presented in this table indicate that the TUNEL analysis detects earlier events in the onset of osteonecrosis than traditional histopathology findings.

TABLE 6
Summary of TUNEL and histopathology analysis

| | | | | Wistar | |
|---|---|---|---|---|---|
| | Histopathology | Fisher rat | | Kyoto rat | |
| | & TUNEL | Placebo | Steroid | Placebo | Steroid |
| Number of hips analysed by | | 9 | 23 | 12 | 22 |
| histopathology | | | | | |
| Number of hips analysed | | 8 | 13 | 7 | 15 |
| by TUNEL | | | | | |
| Left | AVN(+) TUNEL(++) | 1 | 3 | | 3 |
| | AVN(+) TUNEL(+) | | 3 | 2 | 1 |
| | AVN(+) TUNEL(−) | | 1 | | 1 |
| | AVN(−) TUNEL(−) | 2 | | | |
| | AVN(−) TUNEL(+) | 1 | | | 1 |
| | AVN(−) TUNEL(++) | | 2 | 1 | 2 |
| Right | AVN(+) TUNEL(++) | 2 | 1 | 1 | 2 |
| | AVN(+) TUNEL(+) | 1 | 1 | 1 | 2 |
| | AVN(+) TUNEL(−) | | 1 | 1 | |
| | AVN(−) TUNEL(−) | 1 | | 1 | |
| | AVN(−) TUNEL(+) | | | | 1 |
| | AVN(−) TUNEL(++) | | 1 | | 2 |
| Total | TUNEL(++)/TUNEL(−) | 3/3 | 7/2 | 2/2 | 9/1 |

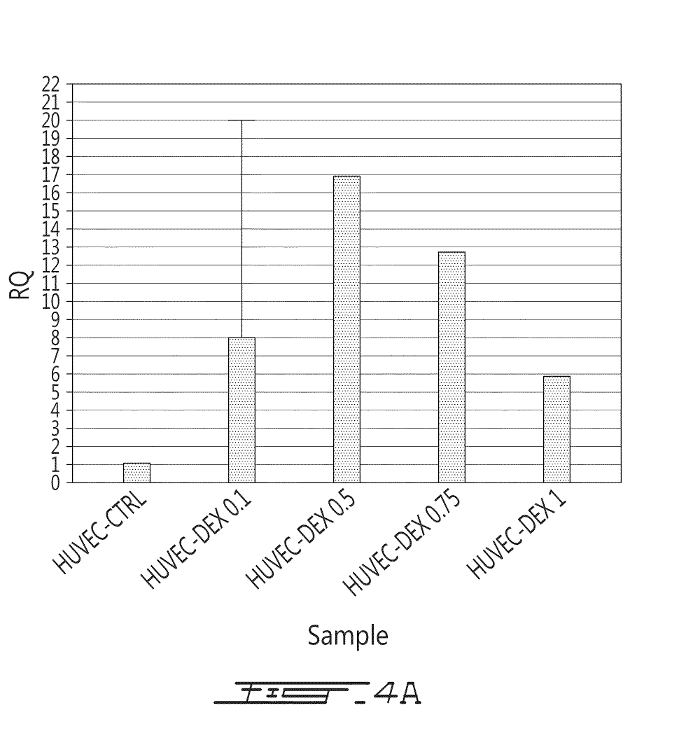

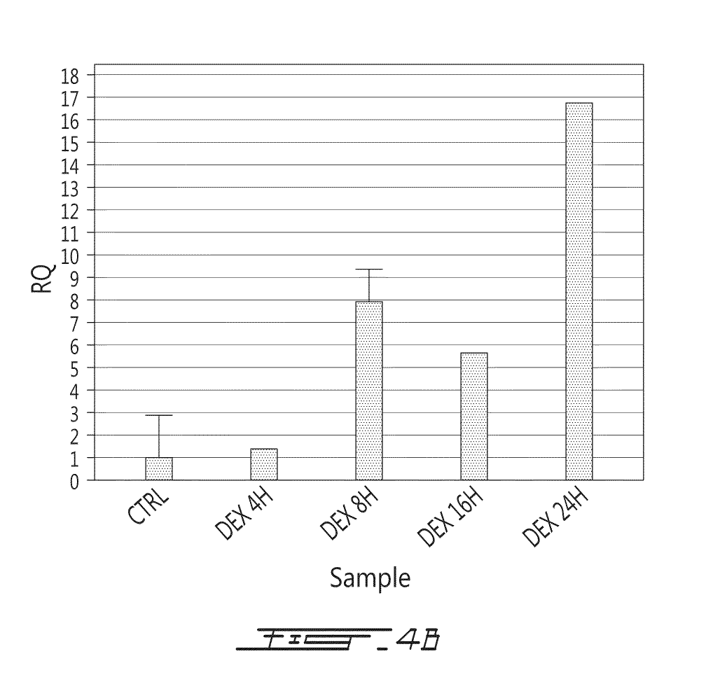

Fisher and Wistar Kyoto (WK) rats received steroid/placebo treatment for 5 and 6 months, respectively and blood was collected monthly. The Alpha-2-Macroglobulin (A2M) level in the blood was detected using rat A2M ELISA kit (Immunology Consultants Laboratory, USA). Rats were divided into groups based on the treatment as well as the results from histopathology and TUNEL evaluation. AVN(+) was judged to be either TUNEL (+) or (++), or AVN(−) by histopathology study on at least one hip. As shown on FIGS. 3A and 3B, the A2M level in the blood from rats treated with placebo (and identified as AVN(+) or AVN (−)) showed no significant difference. However, as shown on FIG. 3C, the A2M level in the blood of steroid-treated Fisher rats (only those considered AVN(+)) increased at 2 months after steroid treatment, and they were consider AVN(+). As shown on FIG. 3D, the A2M level in the blood of steroid-treated WK rats (all considered AVN(+)) reached a peak at 3 month after the beginning of the treatment. As shown in FIG. 3E, in Fisher rats, A2M level declined 5 months after the beginning of the steroid treatment, while in steroid-treated WK rats, the A2M level remained higher than placebo-treated group at least 6 months after the beginning of the treatment.

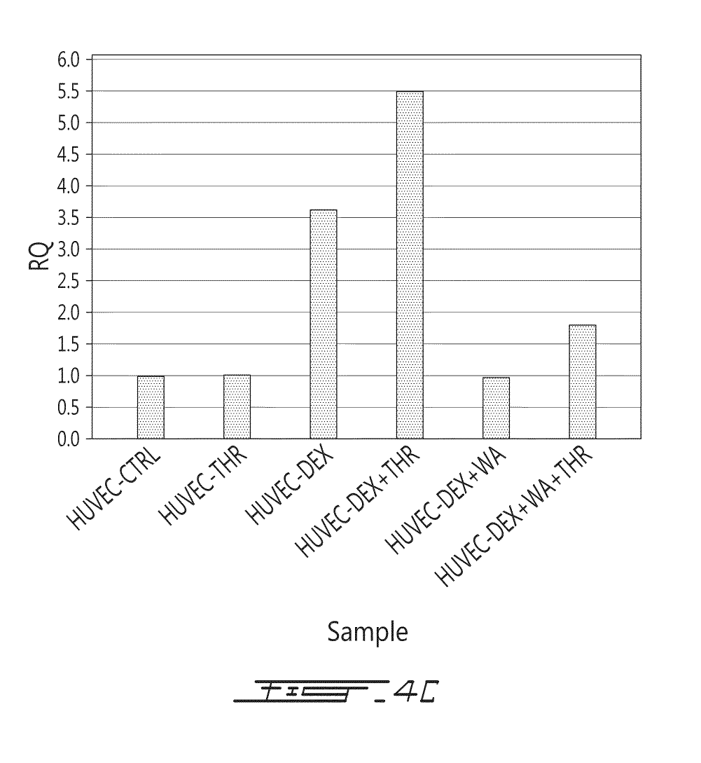

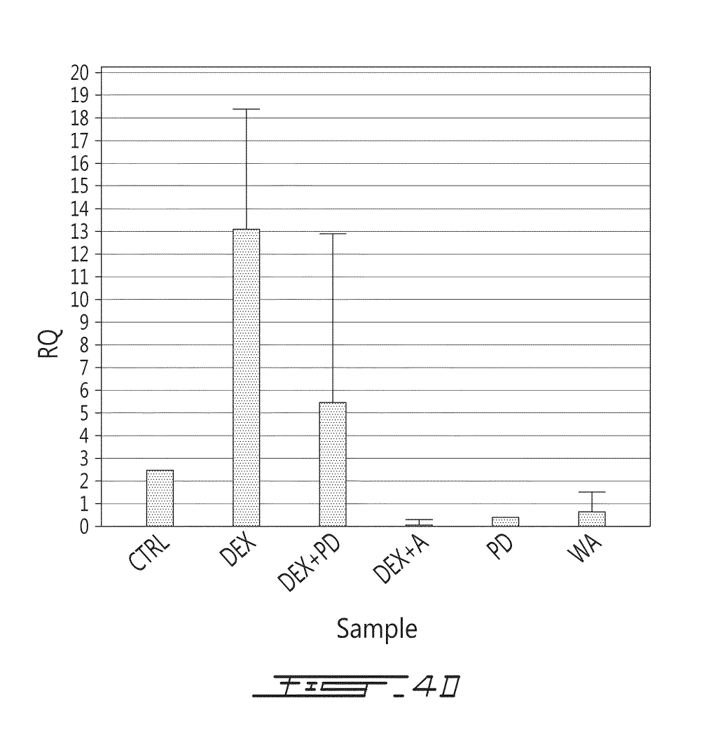

The A2M gene expression in human umbilical vein endothelial cells (HUVEC) treated with various pharmacological agents has also been measured. First, a dose response curve of HUVEC cells treated 48h using different concentration of Dex (dexamethasone) (0.1 mM to 1 mM) was generated. As seen on FIG. 4A, the maximum effect of dexamethasone on A2M expression was observed at 0.5 mM at the experimental conditions tested. Then, a time response curve (4h to 24h) of HUVEC cells treated with 1 mM dexamethasone was generated. As seen on FIG. 4B, the maximum effect of dexamethasone on A2M expression was observed at 24h. The effect of thrombin, dexamethasone and whitaferin on A2M expression of HUVEC cells was then determined. As shown on FIG. 4C, the combination of thrombin and dexamethasone lead to an increase in A2M expression, when compared with thrombin alone or dexamethasone alone. The effect of dexamethasone, PD and whitaferin on A2M expression of HUVEC cells was also determined. As shown on FIG. 4D, A2M overexpression induced by dexamethasone requires MAPK and/or NFκB because inhibition of the MAPK or NFκB pathways induced a repression of A2M gene expression.

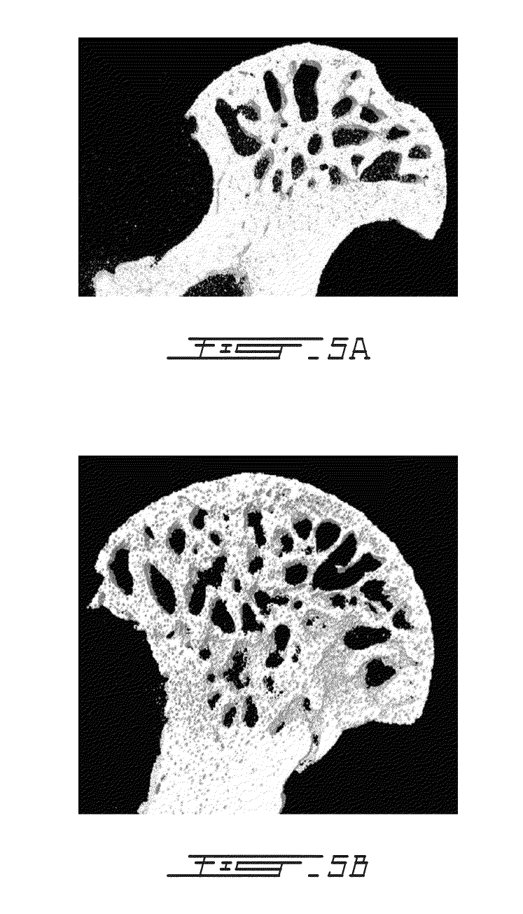

In addition, a micro-computerized tomography scanning of cross-sections of femoral heads was conducted. As presented on FIG. 5A, Fisher rats, even following steroid administration showed no articular erosion, a thick trabeculae as well as well connected trabeculae. As presented on FIG. 5B, 9 out of 12 steroid-treated WK rats showed subchondral porosity, small and erratic trabeculae as well as an isotropic trabecular arrangement.

### REFERENCES

Wada M, Kumagai K, Murata M, Yamashita Y, Shindo H. Warfarin reduces the incidence of osteonecrosis of the femoral head in spontaneously hypertensive rats. J Orthop Sci. 2004; 9: 585-590.
Arlet J. A traumatic necrosis of the femoral head: general report in Bone circulation and vascularization in normal and pathological conditions. New York: Plenum Press; 1993. p. 235-240.
Weinstein R S, Nicholas R W, Manolagas S C. Apoptosis of osteocytes in glucocorticoid-induced osteonecrosis of the hip. J Clin Endocrinol Metab. 2000; 2907-2912.
Shibahara M, Nishida K, Asahara H, Yoshikawa T, Mitani S, Kondo Y, Inoue H.t al. Increased osteocyte apoptosis during the development of femoral head osteonecrosis in spontaneously hypertensive rats. Acta Med Okayama. 2000; 54: 67-74.
Benjamini Y, & Hochberg Y. Controlling the false discovery rate: a practical and powerful approach to multiple testing. Journal of Royal Statistical Society. Series B (Methodological) 1995; 57: 289-300
Weinstein R S, Jilka R L., Parfitt A M, Manolagas S C. Inhibition of osteoblastogenesis and promotion of apoptosis of osteoblasts and osteocytes by glucocorticoids. Potential mechanisms of their deleterious effects on bone. J Clin Invest. 1998; 102: 274-282.
Kabata T, Kubo T, Matsumoto T., Nishino M, Tomita K, Katsuda S, Horii T, Uto N, Kitajima I. Apoptotic cell death in steroid induced osteonecrosis: an experimental study in rabbits. J. Rheumatol. 2000; 27:2166-2171.
Weinstein R S, Nicholas R W, Manolagas S C. Apoptosis of osteocytes in glucocorticoid-induced osteonecrosis of the hip. J Clin Endocrinol Metab. 2000; 85:2907-2912.
Kerachian M A, Cournoyer D, Nahal A, Harvey E J, Chow T Y, Séguin C. Apoptotic cell death in steroid-induced bone disease: A pilot study in rats. Bone. 2007; 40[6]: S287. Ref Type: Abstract

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method of assessing a predisposition to or an affliction by corticosteroid-induced osteonecrosis in an individual, said method comprising:
(a) providing a biological sample from the individual, wherein the individual has received at least a dose of corticosteroid therapy;
(b) combining (i) an antibody specific for at least one biomarker whose expression is positively correlated with corticosteroid-induced osteonecrosis with (ii) the biological sample so as to form a complex between the antibody and the at least one biomarker, wherein said at least one biomarker comprises an alpha-2-macroglobulin (A2M) protein;
(c) measuring an amount of the complex of step (b) to provide a test level of the at least one biomarker whose expression is positively correlated with corticosteroid-induced osteonecrosis;
(d) comparing the test level of step (c) to a control level of the at least one biomarker, wherein the control level is associated with a lack of corticosteroid-induced osteonecrosis; and
(e) characterizing the individual as:
(A) being susceptible to or afflicted by corticosteroid-induced osteonecrosis when the test level is higher than the control level; and
(B) lacking the susceptibility or the affliction to corticosteroid-induced osteonecrosis when the test level is equal to or lower than the control level.

2. The method of claim 1, wherein the at least one biomarker further comprises a collagen type II alpha-1 (col2A1) protein.

3. The method of claim 1, wherein the at least one biomarker further comprises a melanoma inhibitory activity 1 (MIA1) protein.

4. The method of claim 1, wherein the at least one biomarker further comprises at least one of: (i) a steroid stimulus response protein selected from the group consisting of alkaline phosphatase, tissue-nonspecific, transforming growth factor beta 2 and potassium large conductance calcium-activated channel, subfamily m, alpha member 1; and (ii) an apoptosis pathway response protein selected from the group consisting of S100 protein-beta polypeptide, transforming growth factor-beta 2, vitamin D receptor, unc-5 homolog c and growth hormone receptor.

5. The method of claim 1, wherein the at least one biomarker further comprises a protein selected from the group consisting of: scrapie responsive gene 1, growth hormone receptor, SH2B adaptor protein 2, fibromodulin, matrix metallopeptidase 3, proprotein convertase subtilisin/kexin type 6, cadherin 13, calpain 6, murinoglobulin 2, solute carrier family 38, member 3, fibroblast growth factor 1, vitamin D receptor, carbonic anhydrase 8, WNT1 inducible signaling pathway protein 2, integrin binding sialoprotein, calcitonin receptor, S100 protein, beta polypeptide, neural, potassium large conductance calcium-activated channel, subfamily M, alpha member 1, angiopoietin-like 2, pannexin 3, sphingomyelin phosphodiesterase 3, neutral solute carrier family 13 (sodium-dependent citrate transporter) member 5, cadherin 17, unc-5 homolog C, plasminogen activator inhibitor-1, solute carrier organic anion transporter family, member 2a1, melanoma cell adhesion molecule, orosomucoid 1, transforming growth factor beta 2, alkaline phosphatase liver/bone/kidney, basic helix-loop-helix domain containing class B3, immunoglobulin superfamily member 10, transmembrane protein 100, parathyroid hormone receptor 1, a disintegrin-like and metallopeptidase (reprolysin type) with thrombospondin type 1 motif 1, sphingomyelin synthase 2, transient receptor potential cation channel, subfamily V member 4, parvin alpha, regulator of calcineurin 2, latexin, receptor accessory protein 6 and cAMP responsive element binding protein 3-like 1.

6. The method of claim 1, further comprising:
(f) combining (i) an antibody specific for at least one biomarker whose expression is negatively correlated with corticosteroid-induced osteonecrosis with (ii) in the biological sample from the individual so as to form a complex between the antibody and the at least one biomarker, wherein said at least one biomarker is a protein selected from the group consisting of chemokine (C-X-C motif) ligand 13, similar to T-cell receptor alpha chain precursor V and C regions (TRA29), interferon alpha-inducible protein 27-like, RT1-CE13, MAS-related GPR member X2, and/or RT1 class lb gene H2-TL-like grc region (N3);
(g) measuring an amount of the complex of step (f) to provide a test level of the at least one biomarker whose expression is negatively correlated with corticosteroid-induced osteonecrosis;
(h) comparing the test level of step (g) to a control level of the at least one biomarker, wherein the control level is associated with a lack of corticosteroid-induced osteonecrosis; and
(i) characterizing the individual as:
(A) being susceptible to or afflicted by corticosteroid-induced osteonecrosis when the test level is lower than the control level; and
(B) lacking the susceptibility or the affliction to corticosteroid-induced osteonecrosis when the test level is equal to or higher than the control level.

7. The method of claim 1, wherein the individual has received a first dose corticosteroid therapy and the individual is characterized as (i) being susceptible to or afflicted by corticosteroid-induced osteonecrosis when the test level is higher than the control level after the intake of the first dose; and (ii) lacking the susceptibility or the affliction to corticosteroid-induced osteonecrosis when the test level is equal to or lower than the control level after the intake of the first dose.

8. The method of claim 7, wherein the individual has received a second dose of corticosteroid therapy and the individual is characterized as (i) being susceptible to or afflicted by corticosteroid-induced osteonecrosis when the test level is higher than the control level after the intake of the first dose and second dose; and (ii) lacking the susceptibility or the affliction to corticosteroid-induced osteonecrosis when the test level is equal to or lower than the control level after the intake of the second dose.

9. (canceled)

10. The method of claim 1, wherein the biological sample is blood.

11. The method of claim 1, wherein the individual is a human.

12.-21. (canceled)

22. The method of claim 1, wherein the corticosteroid therapy is a glucocorticosteroid therapy.
